(19) United States
(12) Patent Application Publication
(10) Pub. No.: US 2024/0238611 A1
(43) Pub. Date: Jul. 18, 2024

(54) TREATMENT MEDIA DELIVERY DEVICE AND METHODS FOR TARGETED DELIVERY THEREOF

(71) Applicant: W. L. Gore & Associates, Inc., Newark, DE (US)

(72) Inventors: Merrill J. Birdno, Flagstaff, AZ (US); Jared S. Nelson, Flagstaff, AZ (US); Brandon C. Short, Flagstaff, AZ (US)

(21) Appl. No.: 18/567,997

(22) PCT Filed: Jun. 7, 2022
(86) PCT No.: PCT/US2022/032500

§ 371 Date: Dec. 7, 2023

Related U.S. Application Data
(60) Provisional application No. 63/208,436, filed on Jun. 8, 2021.

(51) Int. Cl. A61N 5/06; A61K 9/70; A61K 41/00

(52) U.S. Cl. CPC A61N 5/062; A61K 9/7084; A61K 41/0057; A61N 2005/0602; A61N 2005/063; A61N 2005/0632; A61N 2005/0645; A61N 2005/0661; A61N 2005/0663

(57) ABSTRACT

Described embodiments are directed to a device, system, and methods for delivery of activatable compounds to a patient's tissue. More specifically, described embodiments are directed toward delivery devices operable to deliver the activatable compounds and to activate the compounds. Such delivery devices may include treatment zones for activatable compound delivery and/or activation and non-treatment zones that block activatable compound delivery and/or activation for controlled delivery and activation of the activatable compounds.

### CROSS-REFERENCE TO RELATED APPLICATION

This application is a national phase application of PCT Application No. PCT/US2022/032500, internationally filed on Jun. 7, 2022, which claims the benefit of U.S. Provisional 63/208,436, filed Jun. 8, 2021, which are herein incorporated by reference in their entireties for all purposes.

### FIELD

The present disclosure relates generally to devices and methods for treating biological tissue, such as blood vessels, organs and skin. More specifically, the disclosure relates to treatment delivery system devices and methods for delivering a treatment media to such biological tissue in a targeted manner.

### BACKGROUND

In some applications, the body tissue may be treated to promote various physiological outcomes in the tissue itself. However, the delivery of the treatment to the vessel tissue can be difficult to control. For example, the nature of the vessel can make it difficult to deliver treatment media, such as a therapeutic compound, to a specific site in the vessel because blood flow may flush the therapeutic compound from a selected site, the therapeutic compound may be diluted, or the therapeutic compound may simply be delivered adjacent, rather than directly to, the desired treatment site. For example, during angioplasty procedures, the balloon may present an obstacle to the delivery of therapeutic compounds as the balloon is in contact with the vessel wall at the site that is to be targeted by the treatment. Further difficulties can arise when there is a specific desired treatment that requires multiple steps or elements to make the treatment effective, such as activation of a therapeutic compound at the selected site following delivery of the therapeutic compound.

### SUMMARY

Described embodiments are directed to a device, system, and methods for delivery of activatable compounds to a patient's lumen, such as in transcatheter procedures. More specifically, described embodiments are directed toward delivery devices operable to deliver the activatable compounds and to activate the compounds. Such delivery devices may include treatment zones for activatable compound delivery and/or activation and non-treatment zones that block activatable compound delivery and/or activation for controlled delivery and activation of the activatable compounds.

According to a first example (“Example 1”), a treatment device for delivering a treatment to a lumen of a patient includes a shaft configured to be inserted into the lumen of the patient and a balloon assembly coupled to the shaft is provided. Optionally, the balloon assembly may define a first end, a second end, a first shoulder portion adjacent the first end, a second shoulder portion adjacent a second end, and an intermediate portion located between the first and second shoulder portions, the balloon assembly configured to be inflated from a first size to a second, larger size, wherein the balloon assembly includes a treatment zone, and wherein the balloon assembly is configured to deliver activatable treatment media at the treatment zone of the balloon assembly to the lumen of the patient and to activate the treatment media at the treatment zone of the balloon assembly.

According to another example (“Example 2”), further to Example 1, the treatment device includes a light source in optical communication with the treatment zone of the balloon assembly.

According to another example (“Example 3”) further to Examples 1 or 2, at least a portion of the treatment zone is configured to be light transmissive.

According to another example (“Example 4”) further to Example 3, the portion of the treatment zone configured to be light transmissive has a light transmissivity of at least 40% transmittance over a light wavelength range from 400 nm to 700 nm

According to another example (“Example 5”) further to Examples 3 or 4, the treatment zone includes a hydrophilic material.

According to another example (“Example 6”) further to any of the preceding Examples, the balloon assembly further includes a non-treatment zone of the balloon assembly configured to be light blocking.

According to another example (“Example 7”) further to Example 6, the non-treatment zone includes the first shoulder portion and the second shoulder portion.

According to another example (“Example 8”) further to Examples 6 or 7, the non-treatment zone has a light transmissivity of less than 40% transmittance over a light wavelength range from 400 nm to 700 nm.

According to another example (“Example 9”) further to Examples 6 to 8, the non-treatment zone includes a radio-opaque material.

According to another example (“Example 10”) further to any preceding Example, the balloon assembly includes an expansion layer and a cover layer.

According to another example (“Example 11”) further to Example 10, the balloon assembly further includes an inflation chamber defined within the expansion layer, wherein the inflation chamber is configured to receive a pressurized expansion media for inflating the balloon assembly.

According to another example (“Example 12”) further to Examples 10 or 11, the balloon assembly further includes a delivery chamber located between the expansion layer and the cover layer, wherein the delivery chamber is configured to receive the activatable treatment media for delivery to the lumen of the patient.

According to another example (“Example 13”) further to Example 12, the delivery chamber is fluidically segregated from the inflation chamber.

According to another example (“Example 14”) further to Examples 10 to 13, the expansion layer includes a non-compliant material, a semi-compliant material, a compliant material, or combinations thereof.

According to another example (“Example 15”) further to Examples 10 to 14, the expansion layer includes polyester, nylon, Pebax, polyurethane, silicone, polyethylene or combinations thereof.

According to another example (“Example 16”) further to Examples 10 to 15, the balloon assembly further includes a delivery zone and wherein the cover layer includes a material having porosity configured to weep the activatable treatment media from the delivery zone when the activatable treatment media surpasses a threshold pressure within the delivery chamber.

According to another example (“Example 17”) further to Examples 11 to 16, the shaft includes an inflation conduit in fluid communication with the inflation chamber and a delivery conduit in fluid communication with the delivery chamber.

According to another example (“Example 18”) further to any preceding Example, the treatment device having the activatable treatment media in the form of an extracellular matrix cross-linking promoter that is light activated.

According to another example (“Example 19”) further to any preceding Example, the treatment device having the activatable treatment media in the form of naphthalimide, riboflavin 5′-phosphate, and/or rosebengal.

According to another example (“Example 20”) further to any Example, the shaft further comprises an activation conduit.

According to another example (“Example 21”) further to Example 20, the activation conduit communicates light from the light source to the activation zone of the balloon assembly.

According to an example (“Example 22”), a method of providing treatment to a lumen of a patient's body is provided. The method includes providing a treatment device including a shaft and a balloon assembly coupled to the shaft, the balloon assembly defining a first end, a second end, a first shoulder portion adjacent the first end, a second shoulder portion adjacent a second end, and an intermediate portion located between the first and second shoulder portions, wherein the balloon assembly includes a treatment zone, and is configured to deliver activatable treatment media at the treatment zone of the balloon assembly to the lumen of the patient and to activate the treatment media at the treatment zone of the balloon assembly. The method further includes positioning the balloon assembly inside of the lumen of the patient's body. The method also includes inflating the balloon assembly from a first size to a second, larger size. The method also includes delivering the activatable treatment media to the lumen of the patient at the treatment zone. The method also includes activating the activatable treatment media at the treatment zone of the balloon assembly.

According to another example (“Example 23”) further to Example 22, the treatment device further includes a light source in optical communication with the treatment zone of the balloon assembly, and wherein the step of activating the activatable treatment media includes providing light to the treatment zone of the balloon assembly.

According to another example (“Example 24”) further to Example 23, the activation zone is configured to be light transmissive.

According to another example (“Example 25”) further to Examples 22 to 24, the balloon assembly includes a non-treatment zone that is configured to be light blocking.

According to another example (“Example 26”) further to Examples 22 to 25, the balloon assembly includes an expansion layer and a cover layer, wherein the delivery chamber is located between the expansion layer and the cover layer, and wherein the delivery chamber is fluidically segregated from the inflation chamber.

According to another example (“Example 27”) further to Example 26, the step of inflating the balloon assembly includes providing a pressurized expansion media to the inflation chamber, and wherein the step of delivering the activatable treatment media including providing the activatable treatment media to the delivery chamber.

According to another example (“Example 28”) further to Example 27, the method further includes removing a portion of the pressurized expansion media from the inflation chamber of the balloon assembly to deflate the balloon assembly from the second, larger size to a third size that is greater than the first size and less than the second size prior to providing the activatable treatment media to the delivery chamber.

According to another example (“Example 29”) further to Example 28, the method further includes increasing pressure inside the inflation chamber after providing the activatable treatment media to the delivery chamber, wherein the increasing of the pressure inside the inflation chamber exerts a force on and increases the pressure of the delivery chamber.

According to an example (“Example 30”), a method of manufacturing a treatment device is provided. The method includes preparing a balloon assembly defining a first end, a second end, a first shoulder portion adjacent the first end, a second shoulder portion adjacent a second end, and an intermediate portion located between the first and second shoulder portions, the balloon assembly configured to be inflated from a first size to a second, larger size, wherein the balloon assembly is prepared from a light transmissive material. The method further includes treating the balloon assembly at a predetermined area in order to reduce light transmissivity of the balloon assembly at the predetermined areas, wherein the predetermined area comprises a non-treatment zone and a remaining area of the balloon assembly comprises a treatment zone. The method also includes coupling the balloon assembly to a shaft configured to be inserted into the lumen of the patient.

According to another example (“Example 31”) further to Example 30, the step of treating the balloon assembly at a predetermined area includes masking the predetermined area with a light-blocking material.

According to another example (“Example 32”) further to Example 30, the step of treating the balloon assembly at a predetermined area includes treating the predetermined area with a hydrophilic coating, filler, or adhesive.

According to an example (“Example 33”), a method of manufacturing a treatment device is provided. The method includes preparing a balloon assembly defining a first end, a second end, a first shoulder portion adjacent the first end, a second shoulder portion adjacent a second end, and an intermediate portion located between the first and second shoulder portions, the balloon assembly configured to be inflated from a first size to a second, larger size. The method further includes treating the balloon assembly at a predetermined area in order to increase light transmissivity of the balloon assembly at the predetermined areas, wherein the predetermined area comprises a treatment zone and a remaining area of the balloon assembly comprises a non-treatment zone. The method also includes coupling the balloon assembly to a shaft configured to be inserted into the lumen of the patient.

According to another example (“Example 34”) further to Example 33, the step of treating the balloon assembly at the predetermined area includes providing a plasma treatment to the predetermined area.

According to another example (“Example 35”) further to Example 33, the step of treating the balloon assembly at the predetermined area includes densifying the balloon assembly at the predetermined area.

According to another example (“Example 36”) further to Example 33, the step of treating the balloon assembly at the predetermined areas includes filling pores of the balloon assembly with thermoplastic filler.

According to an example (“Example 37”) further to Example 33, the step of treating the balloon assembly at the predetermined areas includes treating the balloon assembly with hydrophilic material.

According to another example (“Example 38”) further to Example 33, the step of treating the balloon assembly at the predetermined area includes treating the predetermined area with a hydrophilic coating, filler, or adhesive.

According to an example (“Example 39”), a method of providing treatment to a lumen of a patient's body is provided. The method includes positioning a balloon assembly inside of the lumen of the patient's body, the balloon assembly being coupled to a shaft and defining a first end, a second end, a first shoulder portion adjacent the first end, a second shoulder portion adjacent a second end, and an intermediate portion located between the first and second shoulder portions, the balloon assembly including a treatment zone and a non-treatment zone, and being configured to deliver activatable treatment media to the lumen of the patient. The method further includes inflating the balloon assembly from a first size to a second, larger size. The method also includes delivering the activatable treatment media to the lumen of the patient. The method also includes activating the activatable treatment media at the treatment zone of the balloon assembly.

According to another example (“Example 40”) further to Example 39, the step of activating the activatable treatment media includes providing light to the treatment zone of the balloon assembly with a light source in optical communication with the treatment zone of the balloon assembly.

According to another example (“Example 41”) further to Example 39 or 40, the method further includes transmitting light through the treatment zone.

According to another example (“Example 42”) further to Examples 39 to 41, the method further includes blocking transmitted light by a non-treatment zone of the balloon assembly.

According to another example (“Example 43”) further to Examples 39 to 42, where positioning the balloon assembly includes positioning an expansion layer and a cover layer proximate a target site to be treated, wherein the balloon assembly includes a delivery chamber located between the expansion layer and the cover layer, and wherein the delivery chamber is fluidically segregated from an inflation chamber.

According to another example (“Example 44”) further to Example 43, where inflating the balloon assembly includes providing a pressurized expansion media to the inflation chamber, and wherein the step of delivering the activatable treatment media including providing the activatable treatment media to the delivery chamber.

According to another example (“Example 45”) further to Example 44, the method further includes removing a portion of the pressurized expansion media from the inflation chamber of the balloon assembly to deflate the balloon assembly from the second, larger size to a third size that is greater than the first size and less than the second size prior to providing the activatable treatment media to the delivery chamber.

According to another example (“Example 46”) further to Example 45, the method further includes increasing pressure inside the inflation chamber after providing the activatable treatment media to the delivery chamber, wherein the increasing of the pressure inside the inflation chamber exerts a force on and increases the pressure of the delivery chamber.

According to an example (“Example 47”), a method of manufacturing a treatment device including a balloon assembly defining a first end, a second end, a first shoulder portion adjacent the first end, a second shoulder portion adjacent a second end, and an intermediate portion located between the first and second shoulder portions, the balloon assembly configured to be inflated from a first size to a second, larger size, is provided. The method includes configuring a treatment zone of a balloon assembly to be light transmissive and have a first light transmissivity. The method further includes configuring a non-treatment zone of a balloon assembly to be less light transmissive than the treatment zone and have a second light transmissivity that is less than the first light transmissivity.

According to another example (“Example 48”) further to Example 47, wherein configuring the non-treatment zone of the balloon assembly to be less light transmissive than the treatment zone includes providing the non-treatment zone with an outer layer of material having a lower transmissivity than the treatment zone.

According to another example (“Example 49”) further to Example 47 or 48, where configuring the non-treatment zone of the balloon assembly to be less light transmissive than the treatment zone includes providing the non-treatment zone with hydrophobic material.

According to another example (“Example 50”) further to Example 49, where the non-treatment zone is provided with the hydrophobic material by treating the treatment zone with a hydrophobic treatment.

According to another example (“Example 51”) further to Examples 47 to 50, where configuring the treatment zone of the balloon assembly to be light transmissive includes providing the treatment zone with plasma-treated material.

According to another example (“Example 52”) further to Example 51, where the treatment zone is provided with a plasma-treated material by plasma treating the treatment zone.

According to another example (“Example 53”) further to Example 52, where the treatment zone is provided with a plasma-treated material by forming the treatment zone with material that has been plasma-treated.

According to another example (“Example 54”) further to Examples 47 to 53, where configuring the treatment zone of the balloon assembly to be light transmissive includes providing the treatment zone with densified material.

According to another example (“Example 55”) further to Example 54, where the treatment zone is provided with a densified material by using a densification process on the treatment zone.

According to another example (“Example 56”) further to Example 54, wherein the treatment zone is provided with a densified material by forming the treatment zone with material that has undergone a densification process.

According to another example (“Example 57”) further to Example 47 to 56, where configuring the treatment zone of the balloon assembly to be light transmissive includes providing the treatment zone with a hydrophilic material.

According to another example (“Example 58”) further to Example 57, where the treatment zone is provided with a hydrophilic material by coating or filling the treatment zone with hydrophilic material.

According to another example (“Example 59”) further to Example 57, where the treatment zone is provided with a hydrophilic material by forming the treatment zone with material that has been coated or filled with hydrophilic material.

According to an example (“Example 60”), an apparatus for treating a vessel includes an expandable element having at least one opening therethrough, wherein the at least one opening is operable to deliver a light-activatable fluid to the vessel, the expandable element configured to contain and position the light-activatable fluid between the expandable element and the vessel when the expandable element is expanded. The apparatus also includes a light source operable to activate the light-activatable fluid while it is contained and positioned between the expandable element and the vessel.

According to another example (“Example 61”) further to Example 60, the light-activatable fluid promotes scaffold formation on the vessel when activated by light.

According to another example (“Example 62”) further to Example 60 or 61, the expandable element includes a bypass lumen configured to permit blood to flow through the bypass lumen when the expandable element is expanded.

According to an example (“Example 64”), a treatment device for delivering a treatment to a lumen of a patient is provided. The treatment device optionally includes a shaft configured to be inserted into the lumen of the patient and a film forming a balloon assembly coupled to the shaft, the balloon assembly configured to be inflated from a first size to a second, larger size, wherein the balloon assembly includes a treatment zone, and wherein the treatment zone is light transmissive and operable to transfer a treatment media through the film.

According to another example (“Example 65”) further to Example 64, the balloon assembly is at least one layer of expanded polytetrafluoroethylene.

According to another example (“Example 66”) further to Example 64 or 65, the treatment zone of the balloon assembly includes at least one media transfer zone and at least one light transmission zone.

According to another example (“Example 67”) further to Example 66, the at least one light transmission zone is formed of densified expanded polytetrafluorethylene.

According to another example (“Example 68”) further to Example 66 or 67, the at least one media transfer zone is separate from the at least one light transmission zone.

According to another example (“Example 69”) further to Examples 66-68, the at least one media transfer zone is in the substantial shape of a diamond, square, oval, circle, or slit.

According to another example (“Example 70”) further to Examples 66-69, the at least one light transmission zone defines at least 50% of the treatment zone by surface area.

According to another example (“Example 71”) a treatment device for delivering a treatment to a tissue of a patient comprising a patch assembly defining a flat sheet including a fluid delivery system including a delivery chamber defining a wall, the wall including a cover layer defining a planar treatment zone having a porosity operable to allow controlled passage of a fluid from the delivery chamber to an outer surface of the planar treatment zone, wherein the delivery chamber is configured to deliver activatable treatment media to the treatment zone of the delivery chamber to transfer to the tissue of the patient and to activate the activatable treatment media at the tissue.

According to another example (“Example 72”) further to Example 71, further comprising a light source in optical communication with the treatment zone of the delivery chamber.

According to another example (“Example 73”) further to Examples 71-72, wherein at least a portion of the treatment zone is configured to be light transmissive.

According to another example (“Example 74”) further to Examples 71-73, wherein the portion of the treatment zone configured to be light transmissive has a light transmissivity of at least 40% transmittance over a light wavelength range from 250 nm to 700 nm.

According to another example (“Example 75”), further to Examples 71-73 wherein the delivery chamber includes an expansion layer.

According to another example (“Example 76”), further to Example 75, further comprising an inflation chamber adjacent the delivery chamber, the inflation chamber defined within the expansion layer, wherein the inflation chamber is configured to receive a pressurized expansion media for inflating the inflation chamber, wherein inflation of the inflation chamber is operable to urgingly engage the treatment zone against the tissue.

According to another example, (“Example 77”), further to Examples 75 or 76, wherein the delivery chamber is located between the expansion layer and the cover layer, wherein the delivery chamber is configured to receive the activatable treatment media for delivery to the tissue of the patient.

According to another example (“Example 78”), further to Examples 77, wherein the delivery chamber is fluidically segregated from the inflation chamber.

According to another example (“Example 79”), further to Examples 75-78, wherein the expansion layer includes a non-compliant material, a semi-compliant material, a compliant material, or combinations thereof.

According to another example (“Example 80”), further to Examples 75-79, wherein the chamber further includes a delivery subzone and wherein the cover layer includes a material having porosity configured to weep the activatable treatment media from the delivery subzone when the activatable treatment media surpasses a threshold pressure within the delivery chamber.

According to another example (“Example 81”), further to Examples 76-80, wherein the shaft includes an inflation conduit in fluid communication with the inflation chamber and a delivery conduit in fluid communication with the delivery chamber.

According to another example (“Example 82”), further to Examples 71-82, wherein the delivery chamber includes at least one layer of expanded polytetrafluoroethylene, polyethylene, polypelyne, or electrospun PTFE.

According to another example (“Example 83”), a method of providing treatment to a tissue of a patient's body, the method comprising positioning the treatment zone of the treatment device of any of Examples 71-82 against the tissue of the patient's body, delivering the activatable treatment media to the tissue of the patient at the treatment zone, and activating the activatable treatment media at the tissue.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an exemplary treatment device shown being inserted into the lumen of a patient;

FIG. 2 is an exemplary treatment device having a balloon assembly coupled to a shaft in accordance with one embodiment;

FIG. 3 is an exemplary balloon device having treatment and non-treatment zones in accordance with an embodiment;

FIG. 4 is an exemplary balloon device implementing a weep cover in accordance with an embodiment;

FIG. 5 is an exemplary balloon device in which a weep media channel and an inflation media channel are represented in accordance with an embodiment;

FIG. 6 is a shaft having a lumen for light passage running therethrough in accordance with an embodiment;

FIG. 7 is a cross section of a balloon device having a weep media channel, an inflation media channel, and a lumen for light passage disposed in the shaft in accordance with an embodiment; and

FIG. 8 is an exemplary balloon device having a bypass lumen in accordance with an embodiment.

FIG. 9 is an exemplary balloon device having patterned light transmissive zones and fluid transfer zones.

FIGS. 10a and 10b are an exemplary embodiment of a pattern of light transmissive and fluid transfer zones used for a balloon device.

FIGS. 11a and 11b are an exemplary embodiment of a different pattern of light transmissive and fluid transfer zones used for a balloon device.

FIGS. 12-18 are all various embodiments of patterns of light transmissive and fluid transfer zones that may be implemented for a balloon device.

FIGS. 19A-19C are exemplary embodiments of a tissue structure and an exemplary balloon device being implemented in the tissue structure.

FIG. 20 is an exemplary embodiment of a tissue structure shown dissected after delivering a dye to the targeted site.

FIG. 21 is a top view of a patch assembly, in accordance with an embodiment.

FIG. 22 is a cross-sectional view of the patch assembly in accordance with the embodiment of FIG. 21.

FIG. 23 is a top view of a patch assembly on the skin of a patient, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, method steps may be performed in any order or simultaneously, unless it is clear from the context that the method depends on certain actions being performed first.

As used herein, “angioplasty pressure” means the minimum pressure required to perform a PTA procedure for a balloon of a certain size. This value is dependent on the size of the balloon and can be within the working pressure range between the nominal inflation pressure to the rated burst pressure, the nominal inflation pressure being the minimum pressure at which the balloon reaches nominal diameter and rated burst pressure being the upper limit of a pressure range for a medical balloon provided by the manufacturer.

As used herein, “medical device” means any medical device capable of being implanted and/or deployed within a body lumen or cavity. In various embodiments, a medical device can comprise an endovascular medical device such as a stent, stent-graft, graft, heart valve, heart valve frame or pre-stent, occluder, sensor, marker, closure device, filter, embolic protection device, anchor, drug delivery device, cardiac or neurostimulation lead, gastrointestinal sleeve, and the like.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, and may be exaggerated to illustrate various aspects of the present disclosure, and in that regard, the drawing figures should not be construed as limiting.

Various examples relate to treatment delivery system devices and methods for delivering a treatment media to body lumens, such as in association with an angioplasty procedure. In some examples, such systems and methods are configured to provide more precise targeting of a treatment site with enhanced engagement between the area being treated and the balloon from which the treatment is delivered. In some examples, the treatment area/length is less than a total surface area/length represented by a balloon to achieve more robust engagement or contact between a portion of the balloon configured to deliver a treatment media and a vessel wall during delivery of the treatment. In some examples, the treatment media is a collagen cross-linking agent (e.g., light activatable) that helps reinforcement of a tissue wall (e.g., blood vessel) through creating an enhanced collagen matrix. Although treatments discussed herein may contemplate a specific pathophysiology, it is within the scope of the disclosure that the systems and methods may be implemented in various physiologies and for various reasons. By way of example, treatments may include, but are not limited to, dilation of vessels for treatment of plaque formations, durable lumen gain in peripheral artery disease, accelerated A/V fistula maturation, instant fistula maturation, aneurysm endografting seal-zone stabilization, aneurysm growth prevention, thrombus stabilization, dissection repair (e.g., stabilizing dissected segment back into place), perforation repair, venous thrombus reorganization and stabilization, atrial septal defect closure, vascular access site closure, and so forth.

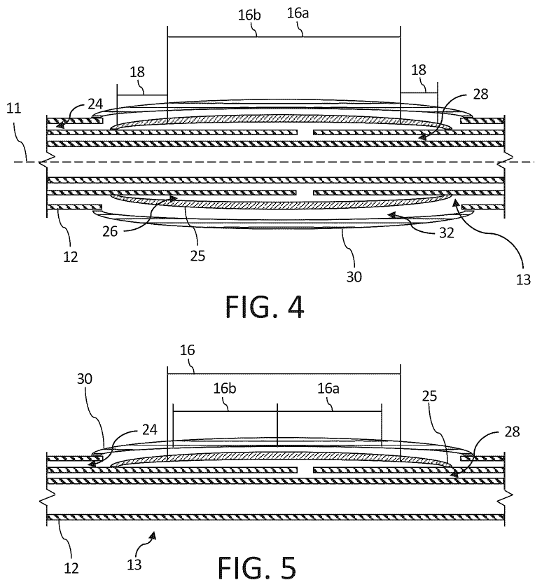

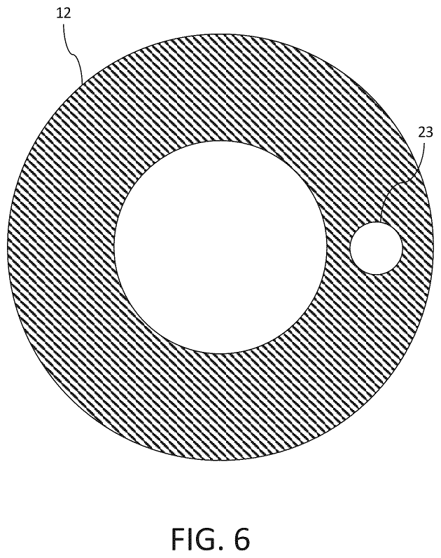

The devices shown in the figures are examples of various device feature and, although the illustrated combinations are clearly within the scope of invention, those examples and their illustrations are not meant to suggest the inventive concepts provided herein are limited from devices with fewer features, additional features, or alternative features to one or more of those features shown in a single figure. As a point of illustration, in various embodiments, the balloon of the device shown in FIG. 4 may include the cover layer described with reference to FIG. 5. And, it should also be understood that the reverse is true as well.

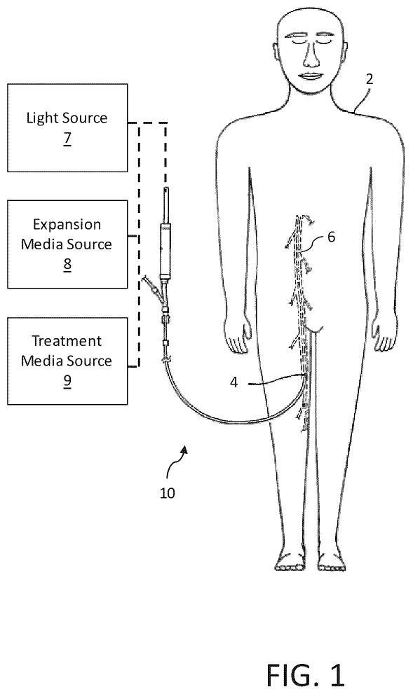

Referring to FIG. 1, the device depicted is a treatment device 10. The treatment device 10 may be inserted into a patient 2 at an insertion site 4. The insertion site 4 allows the treatment device 10 to gain access to a body lumen 6 of a patient 2 (e.g., a vessel). The treatment device 10 may be configured for use in a variety of body lumens, including those of the vasculature, biliary, lymph, respiratory, or gastrointestinal systems, for example. The treatment device 10 may also include a number of features such as a light source 7, an expansion media source 8, and a treatment media source 9. For example, one or all of those features may be incorporated into the treatment device 10 as an integral unit. However, in various embodiments, the treatment device 10 may be operable to connect, couple, or engage with one or more of the light source 7, the expansion media source 8, and the treatment media source 9, which in some embodiments are separate, but connected system components. The treatment device 10 may include a variety of features commonly associated with such assemblies, including one or more handles for user manipulation of the treatment device 10, fluid couplings (e.g., luer fittings), hemostatic valves, and the like.

In some examples, the light source 7 is configured to emit light over a desired wavelength range and at a desired intensity. For example, the light source 7 may be external to the body and light may be carried via fiber optic cable or similar translucent materials. The light source 7 may also be placed in the shaft 12 or inside a balloon assembly such as inside an expansion layer 25, between the expansion layer 25 and a cover layer 30, or woven/attached into the cover layer 30. Light may also be provided by a local light source such as an LED.

In some examples, the expansion media source 8 includes a pressure source (e.g., a manual pump) and an expansion media reservoir (e.g., a container filled with saline). The expansion media source 8 is configured to deliver the expansion media to the treatment device 10 at a desired pressure and may be configured to release or relieve the pressure and/or may be configured to apply a negative pressure in order to carry out an expansion and deflation or retraction cycle. For example, the expansion media source 8 may be comprised of: saline mixed with contrast; saline alone; and/or either of these in combination with the treatment media. The inflation media may be introduced through electromechanical pumps and/or manual methods of pressure adjustments.

In some examples, the treatment media source 9 includes a pressure source (e.g., a manual pump) and a treatment media reservoir (e.g., a container filled with a therapeutic compound). The treatment media source 9 is configured to deliver the treatment media to the treatment device 10 at a desired pressure or flow rate. For example, the treatment media source 9 may be introduced while the expansion pressure is relatively low for angioplasty (≤1 ATM) or while the expansion pressure is sufficient to induce the desired angioplasty effect (3-30 ATM). Though in some examples, the expansion media source 8 and the treatment media source 9 are shown as distinct elements, in some examples the expansion media source 8 and treatment media source 9 are integrated as a single, dual-use system component. For example, the expansion media may serve as a treatment media, and a single pressure source may be used to deliver the combined expansion/treatment media to the treatment device 10.

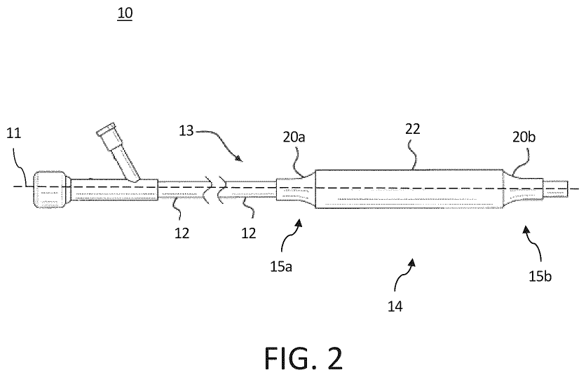

Referring now to FIG. 2, the treatment device 10 includes a shaft 12 that is configured to be inserted into a body lumen 6 of a patient 2 and a balloon assembly 14 coupled to the shaft 12. In use, the balloon assembly 14 is expanded (e.g., to a predetermined diameter) in a body lumen 6 (e.g., blood vessel) of a patient 2 when deployed during a treatment procedure.

The balloon assembly 14 defines a first end 15a, a second end 15b, a first shoulder portion 20a adjacent the first end 15a, a second shoulder portion 20b adjacent the second end 15b, and an intermediate portion 22 located between the first shoulder portion 20a and the second shoulder portion 20b (the first and second shoulder portions 20a, 20b are referred to collectively herein as “shoulders 20”). For reference, the shoulders 20 generally correspond to a taper, or other transition from the main working length of the balloon assembly 14 and the adjacent portions of the shaft 12 to which it is coupled. The shoulders 20 may include a variety of shapes, tapers, steps, contours, overwraps, lengths, and the other features depending on the particular treatment area to which the balloon assembly 14 is being deployed. The treatment device 10 may define a longitudinal axis 11. The shaft 12 may extend along and be disposed about the longitudinal axis 11.

When inflated, the balloon assembly 14 may include shoulders 20 having a diameter less than the diameter of the intermediate portion 22. In other terms, the shoulders 20 acts as a ramping or transition area of the balloon assembly 14 where those portions of the balloon assembly 14 are not at a full size or diameter. Thus, when the balloon assembly 14 is expanded in the patient's lumen (e.g., vessel), the intermediate portion 22 of the balloon assembly 14 is in contact with the vessel wall of the patient, whereas the shoulders 20 may not be in contact or may not have a preferred amount of contact (e.g., relatively continuous engagement and/or with a desired amount of expansion force against the lumen wall) with the vessel wall of the patient.

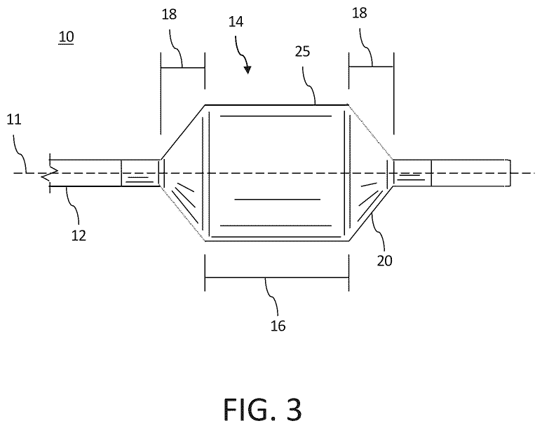

Referring now to FIG. 3, in some embodiments, the balloon assembly 14 includes a first zone and a second zone. The first zone, which can also be described as the treatment zone 16, is a portion of the balloon assembly 14 which is operable to provide the desired treatment at a predetermined position in the body lumen 6. In some examples, the treatment zone 16 includes a selected surface area (e.g., less than an entire outer surface) of the balloon assembly 14. The properties of the treatment zone 16 will be discussed in greater detail hereafter. The second zone 18, which can also be described as a non-treatment zone 18, of the balloon assembly 14 may be configured to be inactive, or to not provide some or all the treatment features exhibited by the treatment zone 16 of the balloon assembly 14. The second zone generally corresponds to one or more surface areas other than those represented by the treatment zone 16 and can be described as the non-treatment zone 18.

In some embodiments, the balloon assembly 14 may include an expansion layer 25. The expansion layer 25 is disposed about the shaft 12 and forms an inflation chamber 26. The inflation chamber 26 may be fully or partially enclosed by the expansion layer 25. The treatment zone 16 and non-treatment zone 18 may be defined on the surface of the expansion layer 25.

In further embodiments, the balloon assembly 14 may comprise an expansion layer 25 and a cover layer 30. The cover layer 30 surrounds or is disposed, fully or in part, around the expansion layer 25. The cover layer 30 may form a delivery chamber 32 radially inward from the cover layer 30. In such examples, the delivery chamber 32 is disposed between the cover layer 30 and the expansion layer 25 and therefore the cover layer 30 is positioned radially outward from the expansion layer 25. The delivery chamber 32 may be described as the space between the interior surface of the cover layer 30 and the exterior surface of the balloon assembly 14, meaning the delivery chamber is a second, separate chamber from the inflation chamber 26.

In some examples, the delivery chamber 32 and the inflation chamber 26 are fluidically separated by the expansion layer 25. Separating the inflation chamber 26 and the delivery chamber 32 permits the inflation chamber 26 and the delivery chamber 32 to be filled with different media (e.g., an expansion media such as saline in the inflation chamber 26 and a treatment media such as the cross-linking agent in the delivery chamber 32). It also permits the inflation chamber 26 and the delivery chamber 32 to be filled and emptied independently. These two features may provide added benefit in combination for improving procedural methods and outcomes when performing an angioplasty procedure. Additionally the delivery chamber 32 may be filled with treatment media, and then delivered by inflating the expansion layer 25 causing an increase in pressure in the cover layer 30 to allow weeping (i.e., using the inflation chamber 26 to provide delivery chamber force), such that there is no need to exchange multiple balloons during the procedure, because one treatment device 10 can provide all of the procedural steps without having to remove or reposition the device 10.

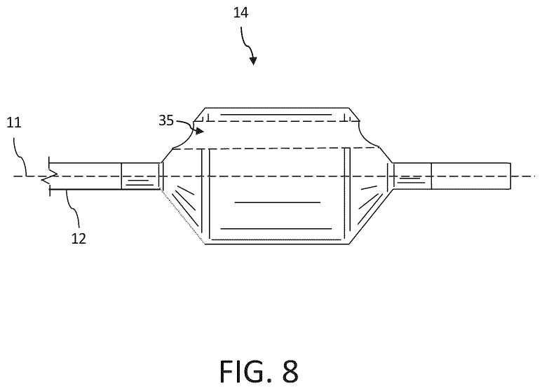

In order to fill and drain the inflation chamber 26 and the delivery chamber 32, the device 10 may include a delivery conduit 24 and an inflation conduit 28. The inflation conduit 28 is in fluid communication with the inflation chamber 26 and the delivery conduit 24 is in fluid communication with the delivery chamber 32. Thus, the delivery chamber 32 and the inflation chamber 26 may be independently filled and emptied. In some embodiments, the delivery conduit 24 and the inflation conduit 28 are disposed in the shaft 12, as seen in FIG. 7. In alternative embodiments, the delivery conduit 24 and the inflation conduit 28 are integrated into the shaft 12 such that each of the delivery conduit 24 and the inflation conduit 28 are disposed about and surrounding the longitudinal axis 11 and run the longitudinal length of the shaft 12, as seen in FIG. 4. Thus, the shaft 12 may carry the expansion media and the treatment media to their respective chambers. The shaft 12, and more specifically the delivery conduit 24 and the inflation conduit 28 are operable to couple to an expansion media source 8 and a treatment media source 9 to allow the media to be distributed to the respective chambers.

Referring to FIG. 4, in order to deliver the inflation and treatment media to the respective chambers, the shaft 12 of the device 10 may further include a stem tube 13. The stem tube 13 includes the portion of the shaft 12 about which the balloon assembly 14 is disposed. Thus, in those embodiments implementing an expansion layer 25 and a cover layer 30, the expansion layer 25 and the cover layer 30 are disposed about the stem tube 13. Likewise, the stem tube 13 may comprise the section of the shaft 12 at which the inflation conduit 28 comes into fluidic contact with the inflation chamber 26 and at which the delivery conduit 24 comes into fluidic contact with the delivery chamber 32.

The treatment zone 16 and the non-treatment zone 18 of the balloon assembly 14 will now be discussed in greater detail. The treatment zone 16 includes a selected surface area (e.g., less than an entire outer surface) of the balloon assembly 14. The non-treatment zone 18 may include the remainder or a portion of the remainder of the outer surface area of the balloon assembly 14. The treatment zone 16 may be subdivided into various sub-zones as well. For instance, the treatment zone 16 may further comprise a delivery subzone 16a and an activation subzone 16b. The delivery subzone 16a and the activation subzone 16b may be represented in adjacent surface areas of the treatment zone 16, may have overlapping areas within the treatment zone 16, or may be coextensive within the treatment zone 16.

The delivery subzone 16a may be characterized by fluid transfer through the balloon assembly 14. This may be achieved in a variety of methods. For example, the balloon assembly 14 may implement various materials including expanded polytetraflouroethylene (“ePTFE”). Various materials such as ePTFE may be configured to incorporate channels or pores which permit fluid to transfer through the cover layer 30 either prior to or upon reaching a specified pressure. This permeability feature facilitates controlled release of the treatment media from the balloon assembly 14. Such channels or pores may be a feature or characteristic of the material itself (e.g., the material microstructure) and/or may be formed during manufacture of the balloon assembly 14 (e.g., via lasing, patterning, and/or etching processes). It will also be understood that fluid transfer may be facilitated by treatments or coatings (e.g., PVA surface treatment for reducing surface tension). The channels or pores may be provided in the delivery subzone 16a with a uniform pattern, a random pattern, or non-uniform patterns, Furthermore, the channels or pores may be provided through the delivery subzone 16a having uniform size and shape of the channels or pores, or the channels or pores may include non-uniform sizes and shapes as desired.

The activation subzone 16b may be characterized by light transmissivity through the balloon assembly 14. In order to achieve light transmissivity, the activation subzone 16b on the balloon assembly 14 may comprise numerous materials and techniques to permit light transmission. For instance, the activation subzone 16b may include a vinyl material. In some examples, vinyl balloon material allows the transmission of an effective amount or dosage of light at predetermined wavelengths (e.g., 470 nm (blue light), 520 nm (green light), 400 nm to 700 nm (all wavelengths of visible light), from 10 nm to 400 nm (ultraviolet light), or infrared light) to transmit through the walls of the balloon assembly 14.

Although vinyl materials may be suitable in various instances, other materials may be employed as desired to achieve a desired light transmission level. For example, ePTFE may be utilized in various examples as a balloon material. The ePTFE may be densified to form the activation subzone 16b to reduce the voids and/or free space in the material to increase the light transmissivity at that portion of the cover layer 30.

Additionally or alternatively, the light transmissivity of the activation subzone 16b may be increased by utilizing hydrophilic properties, such as a hydrophilic film or backer that is configured to wet out to increase light transmissivity (e.g., by reducing surface and/or intralayer voids in the material). In some examples, the activation subzone 16b includes light transmissivity levels of about 70 percent to about 90 percent. Such transmissivity levels may be accomplished utilizing a nylon backer that is properly wetted out. Material wet out may be facilitated by utilizing hydrophilic materials (e.g., hydrophilic coatings or layers) and surface treatments (e.g., plasma treatments). Such hydrophilic materials may include PVA, hydrophilic polymers such as those comprising a PEG chain, hydrogel polymers, Lupasol® SK, Lupasol® WF, and heparin. In addition to plasma treatments, additional material treatments include those utilized to encourage heparin bonding, such as those described in U.S. Pat. No. 9,101,696 to Leontein et al. on Aug. 11, 2015 and U.S. Pat. No. 9,408,950 to Leontein et al on Aug. 9, 2016. In some embodiments, the activation subzone 16b of the balloon assembly may be about 50-90% efficient in light transmissivity. In some embodiments, the activation subzone 16b of the balloon assembly may be from about 20% to about 100% efficient in light transmissivity. In other embodiments, the activation subzone 16b may be about 70-87% efficient in light transmissivity. In other embodiments, the activation subzone 16b may be about 80% efficient in light transmissivity.

Referring to a specific, exemplary embodiment, the balloon assembly 14 may include an expansion layer 25 and a cover layer 30. The layers 25, 30 of the balloon assembly 14 may include a treatment zone 16 with various subzones, including combinations of subzones on each layer. For example, in one embodiment, the treatment zone 16 of the balloon assembly 14 includes an activation subzone 16b on the expansion layer 25 and a delivery subzone 16a on the cover layer 30. The cover layer 30 may also include an activation subzone 16b that is overlapping or coextensive with the delivery subzone 16a.

With regards to the treatment zone 16 of the expansion layer 25, the treatment zone 16 may comprise an activation subzone 16b, which is formed of a vinyl material. As discussed, vinyl may be implemented because of its light transmissive qualities. Furthermore, vinyl (or other sufficiently impermeable material) can helps prevent flow of the pressurized expansion media through the walls of the balloon assembly 14 at operating pressures, while at the same time, allowing transmission of an effective amount or dosage of light at predetermined wavelengths. Thus, the treatment zone 16 of the expansion layer 25 may be configured to be both pressurizable and light transmissive. Stated otherwise, the treatment zone 16 of the expansion layer 25 may be configured to be light transmissive but relatively impermeable to the treatment media under operating conditions.

Regarding the treatment zone 16 of the cover layer 30, the treatment zone 16 may comprise an activation subzone 16b and a delivery subzone 16a. The treatment zone 16 of the cover layer 30 may be formed of ePTFE or other suitable material. In some embodiments, ePTFE provides the appropriate function for a delivery subzone 16a to be included on the cover layer 30 by providing pores formed during the process of rapid expansion. More specifically, ePTFE may be configured to include channels or pores which permit fluid to transfer through the cover layer 30 prior to or after a specific pressure is exerted by the fluid. This allows for the controlled release of the fluid through the cover layer 30. The treatment zone 16 of the cover layer 30 may also permit fluid transfer through the cover layer 30 by the insertion of or creation of channels or pores as discussed previously.

Expanded, or open structure materials such as ePTFE may have relatively low light-transmissivity properties (e.g., due to entrapped air). In order to obtain both light transmissivity and fluid transfer through such materials, one or more portions of the materials may be densified to increase light transmission properties through those portions. Thus, the treatment zone 16 of the cover layer 30 may include light-transmissive characteristics by a combination of procedures including densification of expanded materials, hydrophilic treatments, or others discussed herein. The densification allows the transmission of an effective amount or dosage of light at predetermined wavelengths (e.g., 400-700 nm) to transmit through the walls of the balloon assembly 14.

For example, by densifying selected areas, a pattern of activation subzones 16b and delivery subzones 16a may be created within the treatment zone 16 of the balloon assembly 14 (e.g., with the delivery subzones 16a corresponding to areas having pores or channels for fluid delivery). Such patterning may be uniform or random as desired. For example, a repeating pattern of alternating rings, or circumferential areas corresponding to alternating, ring-shaped activation and delivery zones may be implemented. Thus, the treatment zone 16 of the cover layer 30 can be configured to be both light-transmissive and permeable to the treatment media under operating conditions (e.g., at a selected treatment delivery pressure).

Now turning to the non-treatment zone 18 of the balloon assembly 14, various materials and methods may be implemented to achieve the desired characteristics to limit or prevent, at least in part, the treatment of the body lumen 6 (e.g., vessel tissue) outside of the desired area to be targeted. The non-treatment zone 18 may include a masked or masking zone. For example, referring to FIG. 3, the shoulders 20 of the balloon assembly 14 may comprise, in part or in whole, a non-treatment zone 18 of the device 10. In some embodiments, the shoulders 20 do not have preferred contact with the vessel wall when the balloon assembly is at angioplasty pressure, and therefore treatment may not be desired for this portion of the vessel wall. Thus, the shoulders 20 may include predetermined properties which do not permit treatment and are therefore part of the non-treatment zone 18.

The non-treatment zone 18 may be configured to limit or avoid delivery of treatment media, activation of the treatment media, or both. For example, the non-treatment zone 18 may be masked to prevent or limit light and/or treatment media from passing through the non-treatment zone 18 of the expansion layer 25 or the cover layer 30. The masking may include a film or foil that is attached to the balloon assembly 14 or the cover layer 30 at the non-treatment zone 18, wherein the film or foil is non-light-transmissive or light-blocking and/or non-permeable at operating pressures. Activation blocking (e.g., light blocking) features may be a result of certain adhesives, additives, dyes, or pigments that are incorporated into the expansion layer 25 and/or the cover layer 30 at the non-treatment zone 18. Radio-opaque materials, additives, fillers, or powders may include tantalum (or tantalum oxide), titanium, gold, platinum, or carbon.

Another method for preventing the transmission of light through the non-treatment zone 18 of the device 10 includes preparing the non-treatment zone 18 the balloon assembly 14 and/or the cover layer 30 using air entrapment. This can be accomplished in a variety of ways known to one of skill in the art, including incorporating expanded materials or other materials with microstructures that entrap sufficient air to impede light transmission. Hydrophobic properties can be utilized such as hydrophobic coatings, fillers, or adhesives on the non-treatment zone 18. This can help prevent the non-treatment zone 18 from being wetted and therefore restricts or reduces the light transmissivity at those areas. When the non-treatment zone 18 includes prevention or limitation of fluid transfer through the balloon assembly 14, the prevention or limitation of fluid transfer may be a result of the materials used to form the non-treatment zone 18. For example, a non-permeable material might include vinyl. Fluorinated ethylene propylene (“FEP”) may also be implemented to prevent fluid transfer or weeping in the non-treatment zones 18. Any number of coatings, films, or adhesives may be added to the non-treatment zones 18 in order to help prevent fluid transfer through the balloon assembly 14 and/or the cover layer 30, for example those discussed above.

As some embodiments described herein require activation of a therapeutic benefit, this will be described in more detail, including additional components that may be included on the treatment device 10. In those embodiments where light activation is necessary for activation of the therapeutic compound, the treatment device may include light transmission capabilities. This can include light source 7 being integrated into the device 10 or being coupled to the device 10.

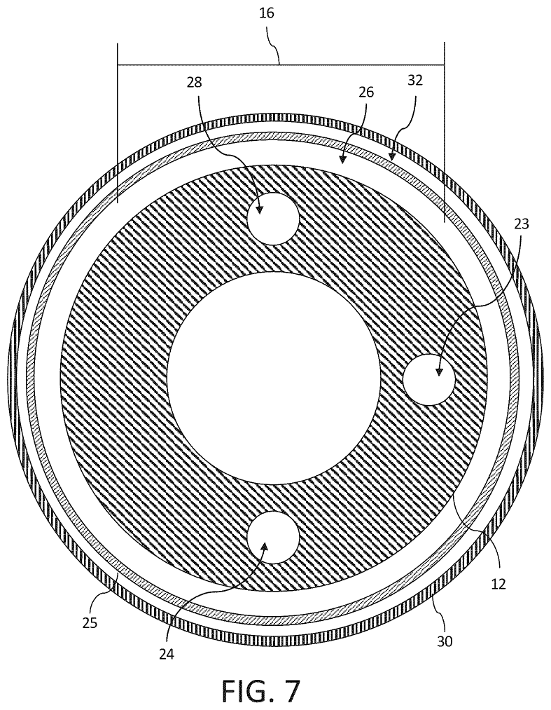

In one embodiment as shown in FIG. 6, the shaft 12 may include a lumen for light passage 23 for transmitting light from one portion of the shaft 12 to another. For example, the lumen for light passage 23 can transmit light from a handle-end of the treatment device 10, along the shaft 12, and to the stem tube 13, which includes the balloon assembly 14. The lumen for light passage 23 may open at the stem tube 13 such that light is emitted near the balloon assembly 14. The stem tube 13 may include a transparent polymer or other transparent material through which light may be transmitted from the end of the lumen for light passage 23. Thus, the light can be transmitted through the cover layer 30 and/or the expansion layer 25 for activating the therapeutic compound. In some embodiments, the stem tube 13 may extend down the longitudinal length of the shaft 12.

Light may be emitted from the lumen for light passage 23, through the treatment zone 16, and more specifically the activation subzone 16b of the balloon assembly 14, and onto the vessel wall. When the light-activatable therapeutic compound that is in contact with and has permeated the tissue of the vessel wall is activated by the light, the therapeutic function is imparted. The non-treatment zone 18 is configured to prevent or reduce transmission of light relative to the treatment zone 16 and consequently prevents or reduces permeation of the light into the vessel wall, which leaves the therapeutic compound inactivated.

In other embodiments, the light source 7 is coupled directly to the device 10 at, near, or with the balloon assembly 14 and the stem tube 13. For example, the light source may be embedded in the stem tube 13 and is thus able to emit light directly from the stem tube 13 through the treatment zone 16 of the device 10. In some embodiments, the device 10 may include a lighted film that is applied at or near the treatment zone 16 of the balloon assembly 14 and/or the cover layer 30.

Although the specific examples of the treatment zone 16 and non-treatment zone 18 provided have included light transmissivity and fluid permeability of the balloon assembly 14, these examples are not limiting. For example, other features that might be incorporated into the treatment zone 16 and non-treatment zone 18 of the balloon assembly 14 might include conductivity, selective permeability, ultrasound-capabilities, resonance, magnetic or ferro-magnetic properties, as well as others.

As the described, in various examples the device is operable for use in surgical procedures, and more specifically transcatheter surgical procedures. For example, the treatment device 10 may be used in connection with an angioplasty procedure for treating a body lumen 6 (e.g., a vessel) of a patient 2. A portion of the device 10 including the shaft 12 and the balloon assembly 14 may be introduced into a body lumen 6 of the patient at the insertion site 4. The treatment device 10 may then be advanced in the patient's vasculature until the balloon assembly has reached a target area of the body lumen 6. The target area of the body lumen 6 may include diseased or damaged vessel walls or an accumulation of plaque on the vessel walls resulting in a buildup, occlusion, or partial occlusion over time. The device 10 is operable to deliver a treatment to the body lumen 6 to correct or treat the physiological or anatomical issue.

Referring to one exemplary angioplasty treatment, an occluded vessel may be expanded by the balloon assembly 14 when the balloon assembly 14 is inflated to an angioplasty pressure, wherein the balloon assembly 14 contacts the vessel wall to widen the narrowed vessel. The balloon assembly 14 then releases a therapeutic compound either along the length of the balloon assembly 14 or alternatively in the treatment zone 16 of the balloon assembly 14. As the therapeutic compound is released from the balloon, it is delivered directly to the vessel wall. The therapeutic compound may include an extracellular matrix (e.g., collagen and/or elastin) cross-linking agent to reduce the propensity of the vessel to return to its pre-treatment diameter and may be delivered in or as a treatment media. Otherwise, in absence of further intervention beyond inflation of the balloon assembly, the vessel's plastic or elastic nature may return the vessel toward its prior shape after the balloon assembly 14 has been deflated and is not providing the force against the vessel wall without some other reinforcement. The treatment media may be delivered from the balloon assembly 14 while the balloon assembly 14 is engaged with the vessel wall.

In order to deliver the treatment media with the therapeutic compound in a controlled manner, a cover layer 30 may be implemented in combination with an expansion layer 25. The expansion layer 25 may be inflated to angioplasty pressure by providing pressurized expansion media to the inflation chamber 26. Referring to a portion of the above method, by permitting the inflation chamber 26 and the delivery chamber 32 to fill and drain independently, a surgeon may be able to first move the device 10 into the appropriate position near the occlusion of the vessel. The inflation chamber 26 may be filled with an inflation media such as a saline solution to angioplasty pressure such that the device 10 is imparting a force against the vessel wall and the occlusion. The saline solution or pressurized expansion media may include a contrast such as a dye, radio-opaque material, or otherwise detectable material for positioning the balloon assembly 14 during the angioplasty procedure. This allows the surgeon to both position the device 10 and to ensure that when the balloon assembly 14 has been inflated that the contact and fit within the vessel is acceptable.

Once the desired placement and fit has been achieved within the vessel, the delivery chamber 32 may be filled with a pressurized treatment media which is then transferred through the cover layer 30. However, in some embodiments, the surgeon may slightly drain or remove expansion media from the inflation chamber 26. This may create space for the filling of the delivery chamber 32 of the cover layer 30. The delivery chamber 32 may be filled with a treatment media, which will weep or transfer through the cover layer 30 when the delivery chamber 32 is at or above a predetermined pressure. If the delivery chamber 32 does not reach the appropriate pressure for a pressure differential to allow the pressurized treatment media to pass through the cover layer 30, the expansion layer 25 may be further inflated to increase the pressure in the delivery chamber 32 in a controlled manner. Thus, the pressurized treatment media may have a controlled release through the filling and draining of both the delivery chamber 32 and the inflation chamber 26. This process can be sustained and repeated to provide sustained release and deeper penetration of the vessel tissue. It may be undesirable in some embodiments for the inflation media to be released into the delivery chamber 32 and/or the patient's vessel, either for dilution of therapeutic compounds or for adverse interactions either with the therapeutic compounds or with the patient's body. Thus, it may be preferable in some embodiments to ensure that the expansion layer 25 is non-permeable under operating conditions to help prevent or reduce the incidence of fluid transfer between the inflation chamber 26 and the delivery chamber 32.

In those embodiments in which the treatment media must be activated, such as by light (e.g., naphthalimide, riboflavin 5′-phosphate, or rosebengal), the method may include a step of providing light to the treatment media. This may be accomplished by providing light to the device 10 from a light source 7. The light is transmitted through the lumen for light passage 23, through the treatment zone 16, and specifically the activation subzone 16b as previously discussed, and to the treatment media which is then activated.

In some embodiments the saline with contrast may be further diluted in order to increase the light transmissivity of the inflation media to about 50-90% efficiency, 70-87% efficiency, or about 80% efficiency.

It will be recognized that a variety of therapies can be delivered to the vessel wall, and therefore those discussed herein are not to be construed as limiting the types of therapies that may be utilized in connection with the devices and methods described herein.

In an embodiment, the therapeutic compound includes a drug which promotes the bonding of tissue and the cross-linking of collagen in tissue, such as described in U.S. Pat. No. 7,514,399 to Utecht et al. on Apr. 7, 2009, U.S. Pat. No. 8,242,114 to Utecht et al. on Aug. 14, 2012, U.S. Pat. No. 8,546,384 to Utecht et al on Oct. 1, 2013, U.S. Pat. No. 8,632,565 to Utecht et al., U.S. Pat. No. 8,741,270 to Utecht et al. on Jun. 3, 2014, U.S. Pat. No. 9,125,938 to Utecht et al. on Sep. 8, 2015, U.S. Pat. No. 9,822,189 to Utecht et al. on Nov. 21, 2017, and U.S. Pat. No. 10,053,521 to Utecht et al. on Aug. 21, 20181. Other therapies may be delivered, including plaque softening agents, such as described in U.S. Pat. No. 10,131,635 to Haberer et al Nov. 20, 2018. For example, the device 10 may provide a therapeutic compound which promotes cross-linking of collagen in the native tissue, wherein the therapeutic compound is activated by predefined wavelengths of light.

In those embodiments where light transmission is necessary for activation of the therapeutic compound, the treatment zone 16 and non-treatment zone 18 may be differentiated by light transmissivity. Although the tissue may be exposed to the therapeutic compound outside of the contact area of the treatment zone 16, without activation of the therapeutic compound the tissue may not receive therapeutic benefit because activation of the therapeutic compound is generally limited to the contact area of the treatment zone 16 due to light transmission. Thus, activation is most pronounced in the treatment zone 16. Therefore, in some embodiments, fluid transmission may not be restricted only to the treatment zone 16 of the device 10. For example, in those embodiments implementing a cover layer 30, the entire surface area of the cover layer 30 may be fluid-permeable at a predetermined pressure gradient which allows the weep media or therapeutic compound to transfer through the cover layer 30 and to contact and permeate the surrounding tissue before, at, or above a predetermined pressure.

However, in some embodiments it may be desirable to limit fluid transfer to specific areas or zones of the balloon assembly 14, such as the treatment zone 16 of the balloon assembly 14. The non-treatment zone may include a non-permeable material, coating, or treatment which restricts or limits the permeability of the non-treatment zone 18. For example, the shoulders 20 of the balloon assembly 14 may comprise a non-treatment zone 18. The limitation of fluid transfer may be important in various embodiments in which the therapeutic compound is active regardless of light activation. Thus, in those embodiments, the treatment zone 16 may be specifically characterized by fluid transfer and the non-treatment zone 18 may be characterized by non-fluid transfer or limited fluid transfer.

In those embodiments in which the balloon assembly includes an expansion layer 25 and a cover layer 30, the treatment zone 16 may include a selected surface area across the expansion layer 25 and the cover layer 30. The non-treatment zone 18 may also be included on both the balloon assembly 14 and the cover layer 30 on areas or surfaces not represented by the treatment zone 16. Such areas including a non-treatment zone as discussed previously might include the shoulder 20 of the balloon assembly 14. The cover layer 30, which in some embodiments, encompasses the balloon assembly 14, may include a similar form factor as the expansion layer 25, such as shoulders 20 and a body.

Furthermore, in some embodiments implementing an expansion layer 25 and a cover layer 30, the balloon assembly 14 may include a treatment zone 16 on the expansion layer 25 and a treatment zone 16 on the cover layer 30. Because the expansion layer 25 and the cover layer 30 may serve different functions, in some embodiments, the treatment zones 16 of the expansion layer 25 and the cover layer 30 may have different features. To provide further reference, the treatment zones 16 may provide various and varying functionalities. The varying features may be described as sub-zones as previously discussed. The sub-zones may represent various features within the treatment zone 16, for example the treatment zone 16 of the cover layer 30 may include an activation subzone 16b and a delivery subzone 16a, whereas the treatment zone 16 of the expansion layer 25 may include an activation subzone 16b but not a delivery subzone 16a.

Although a specific implementation has been provided as an example with regards to the balloon assembly 14 and treatment zones 16 with sub-zones, the disclosure is not to be limited to the specific combination provided. In those embodiments in which light is transmitted through the balloon assembly 14, the materials implemented may vary with regards to both the cover layer 30 and the expansion layer 25.

In a more specific example, when the device 10 includes an expansion layer 25 and a cover layer 30, the treatment zone 16 on the expansion layer 25 may include an activation subzone 16b that is light-transmissive and the corresponding treatment zone 16 on the cover layer 30 also includes an activation subzone 16b that is light-transmissive. However, the treatment zone 16 of the expansion layer may prevent fluid transfer whereas the corresponding treatment zone 16 of the cover layer 30 includes a delivery subzone 16a which permits fluid transfer. Thus, the cover layer 30 may permit the fluid transfer including the activatable treatment media, wherein the activatable treatment media may be activated by light that passes through both the expansion layer 25 and the cover layer 30. For example, the activatable treatment media may include a therapeutic compound that promotes the cross-linking of collagen. These examples are only provided for reference, and it will be noted that the treatment zone 16 may include an assortment of sub zones, including but not limited to an activation subzone 16b and a delivery subzone 16a.

In order to facilitate the activation of the treatment for cross-linking collagen in the tissue, the device 10 may include light transmission capabilities. This can include a light source directly on the device, or which can be coupled to the device. In one embodiment as shown in FIGS. 6 and 7, the shaft 12 may include a lumen for light passage 23 for transmitting light from one portion of the shaft 12 to another. For example, the lumen for light passage 23 can transmit light from a handle-end of a catheter deployment device, along the shaft 12, and to the stem tube 13, which can include the cover layer 30 and/or the balloon assembly 14. The lumen for light passage 23 may open at the stem tube 13 such that light is emitted near the balloon assembly 14. The stem tube 13 may include a transparent polymer or other transparent material through which light may be transmitted from the end of the lumen for light passage 23. Thus, the light can be transmitted through the cover layer 30 and/or the expansion layer 25 for activating the therapeutic compound. In some embodiments, the stem tube 13 may extend down the longitudinal length of the shaft 12.

In those embodiments in which the device 10 includes a treatment zone 16 and a non-treatment zone 18, the light is emitted from the lumen for light passage 23, through the treatment zone 16 of the cover layer 30 and/or the expansion layer 25, and onto the vessel wall. When the therapeutic compound that is in contact with and has permeated the tissue of the vessel wall is activated by the light, the compound promotes the cross-linkage of vessel collagen. The non-treatment zone 18 is configured to prevent or reduce transmission of light relative to the treatment zone 16 and consequently permeation of the light into the vessel wall, which leaves the therapeutic compound inactivated and therefore does not promote collagen cross-linking or does so to a lesser degree.

In other embodiments, the light source is coupled directly to the device 10 at the opposite end including the balloon assembly 14 and the stem tube 13. For example, the light source may be embedded in the stem tube 13 and is thus able to emit light directly from the stem tube 13 through the treatment zone 16 of the device 10. In another alternative embodiment, the device 10 may include a lighted film that is applied at or near the treatment zone 16 of the balloon assembly 14 and/or the cover layer 30.

Various alternative embodiments are within the scope of this disclosure and will be discussed herein. As shown in FIG. 4, the balloon assembly 14 and the cover layer 30 may be disposed about and surround the stem tube 13. In an alternative embodiment as show in FIG. 5, the balloon assembly 14 and cover layer 30 may be only partially disposed about the stem tube 13. This permits the treatment to be applied only to a specific region of the vessel wall. This may be alternatively accomplished by the device 10 by providing a treatment zone 16 on the cover layer 30 and/or the expansion layer 25 in longitudinal strips. In this embodiment, the cover layer 30 and the expansion layer 25 are disposed about the entire circumference of the stem tube 13 and the treatment zones 16 are positioned along the longitudinal length of the cover layer 30 and expansion layer 25. Thus, non-treatment zones 18 are likewise positioned along the longitudinal length of the cover layer 30 and the balloon assembly 14, where the non-treatment zone 18 is positioned at a different arc-length of the device 10 relative to the treatment zone 16.

It will be noted that in some embodiments, the cover layer 30 may comprise a tear-away cover. The tear-away cover may be implemented in those embodiments in which the tear-away cover does not include a treatment zone 16. For example, the balloon assembly 14 may be used in connection with an angioplasty procedure to deliver an activatable therapeutic compound to the vessel of a patient. The therapeutic compound in this example is activatable by light. The tear-away cover may allow for the device 10 to weep the therapeutic compound into the vessel of the patient, however it is not light transmissive. The tear-away cover may be removed from the balloon assembly 14 in order for light to be transmitted to the vessel of the patient in order for the therapeutic compound to be activated and to impart the benefit.

In some embodiments, a conformable balloon assembly may be utilized in connection with the device 10. The conformable balloon assembly may include a latex material that is compliant and may include a film cover. In other embodiments, the device 10 may include a cover layer 30 that is used to transfer a therapeutic compound to the vessel and is not supported or used in conjunction with a balloon assembly.

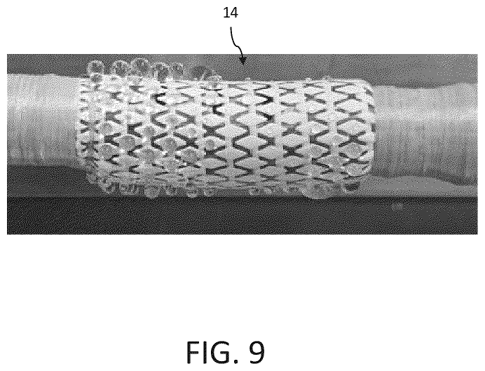

In other embodiments as seen in FIG. 8, the balloon assembly 14 may be formed to include an opening or a bypass lumen 35 in the center or otherwise that allows blood to flow through the balloon assembly 14 while the outer portion of the balloon assembly 14 is in contact with the wall of the artery and is delivering the therapy. The bypass lumen 35 may be sealed such that the blood flowing through the aperture does not have contact with surfaces of the balloon assembly 14 that release a therapeutic compound. Thus, the bypass lumen 35 may run near or substantially parallel to the longitudinal axis 11, such that the balloon assembly 14 does not disrupt or occlude flood flow through the vessel when deployed.

In those embodiments that include only a balloon assembly 14 and no cover layer, the pressurized expansion media may include a therapeutic compound and the balloon assembly 14 is operable to transfer fluid through the balloon assembly 14 in order to treat the vessel. In this embodiment, the balloon assembly 14 comprises an expansion layer 25. The expansion layer 25 may include a treatment zone 16 and a non-treatment zone 18. The treatment zone 16 may include those features as described herein such as a delivery subzone 16a and an activation subzone 16b. The expansion layer 25 may be inflated by a pressurized treatment media being introduced into the inflation chamber 26. The expansion layer 25 may contact the walls of the vessel and begin to distribute or weep the treatment media through the expansion layer 25 once the inflation chamber 26 reaches or surpasses a predetermined pressure. Optionally, after the treatment media is delivered to the vessel wall, the treatment media remaining in the balloon assembly may be withdrawn from the balloon assembly and replaced with an inert fluid, such as, but not limited to saline, prior to light activation. Withdrawing the treatment media from the balloon assembly may be beneficial for, by way of example, but not limited thereto, preventing activation within the balloon assembly and better light transmissibility relative to the treatment media. The treatment device 10 may then be activated to emit light waves through the treatment zone 16 in order to activate the therapeutic compound of the treatment media that was delivered to the vessel wall. Although a specific embodiment of a treatment device 10 having an expansion layer 25 and no cover layer was described in a specific embodiment, it will be recognized that the various features disclosed herein may be implemented and combined with reference to this specific embodiment.

In some embodiments, portions of the balloon assembly 14 of the treatment device 10 may be formed as a composite of a plurality of layers, such as of an inner composite balloon layer and an outer composite balloon layer. In one embodiment, a cover layer may include an inner composite balloon layer and an outer composite balloon layer. The inner composite balloon layer may, for example, be a porous retracted membrane with bent or serpentine fibrils and an optionally composite material, such as an elastomeric coating or filler. The inner composite balloon layer may be helically wrapped or may take other forms, such as concentrically wrapped or extruded forms. The inner composite balloon layer may implement a material selected and/or modified to permit transfer of fluid therethrough under predetermined conditions. For example, the inner composite balloon layer may be treated or otherwise modified to facilitate transfer of the treatment media across the membrane at predetermined pressures or pressure ranges. Pressures or pressure ranges at which the disclosed constructions transfer fluid or weep include from about 1 atm to about 100 atm, from about 2 atm to about 50 atm, from about 2.5 atm to about 20 atm, from about 3 atm to about 10 atm, at about 3 atm, at about 4 atm, at about 5 atm, at about 6 atm, at about 7 atm, at about 8 atm, at about 10 atm, at about 12 atm or other pressures determined by the material and material selection. In some embodiments, the inner composite balloon layer may be selected for the pressures at which fluids transfer through the membrane. The inner composite balloon layer includes at least one layer of wrapped, extruded and/or molded materials (e.g., a film). In some embodiments, the inner composite balloon layer includes multiple layers of extruded and/or molded materials wrapped together, for example, 2-100 film layers. In some embodiments, the inner composite balloon layer includes from about 2 to about 75 layers, from about 2 to about 20 layers, and from about 2 to about 10 layers. In some embodiments, the inner composite balloon layer may be limited to about 2-4 layers, thus permitting high levels of light transmittance through the inner composite balloon layer. High levels of light transmittance may be from about 20% to about 100%. In some embodiments, the inner composite balloon layer may be limited to about 10-40 layers. The light transmissivity of the inner balloon layer may be adjusted via material choice, thickness of the material used for wrapping, number of material layers used in construction, and/or treatment of the material layers prior to, during, or after manufacture (e.g., film densification and/or treatments for enabling wetting out). In some embodiments, the inner composite balloon layer may permit at least 40% light transmittance. In some embodiments, the inner composite balloon layer may permit at least 60% light transmittance. In some embodiments, the inner composite balloon layer may permit at least 80% light transmittance. It will be noted that the inner composited balloon layer may not have any specific strength requirements. Thus, in these embodiments, the inner composite balloon layer may define both a delivery subzone 16a and an activation subzone 16b coextensively.

The outer composite balloon layer may include a material or materials permitting at least 20% light transmittance, thus allowing high transmittance of light through the outer composite layer. In some embodiments the outer composite balloon layer is formed of non-porous, thin, and/or dense material (e.g., expanded materials that have been densified, including expanded fluoropolymers such as ePTFE with or without secondary components including imbibed and/or coated fluoroelastomers). The outer composite balloon layer may be helically-wrapped or concentrically-wrapped, for example, or may be formed using other methods such as extrusion or molding. Small holes or apertures may be formed through the outer composite balloon layer in order to provide porosity or forced porosity. The holes may be formed in any manner, including, but not limited to, drilling or laser cutting. The holes do not need to be tuned for specific water entry pressure (“WEP”) performance, as the inner composite balloon layer controls the WEP performance. The outer composite balloon layer is selected and/or modified for facilitating control of the diameter of the balloon and its strength without risk of introducing porosity under the loads achieved during a procedure (porosity induces opacity and thus obstructs or filters light). Thus, in these embodiments, the outer composite balloon layer may define both a delivery subzone 16a and an activation subzone 16b coextensively.

In some embodiments, the outer composite layer may include a plurality of layers of expanded materials having retracted microstructures (e.g., bent or s-shaped fibrillated structures), including expanded retracted fluoropolymers such as ePTFE with or without secondary components (e.g., imbibed and/or coated fluoroelastomers) helically wrapped, or may take other forms, such as concentrically wrapped or extruded forms. In some embodiments, the outer composite layer may be formed or constructed similar to conformable balloons, such as those described in U.S. Pat. No. 10,076,642 to Campbell et al on Sep. 18, 2018. The plurality of layers may be construction tuned to control the diameter and strength of the balloon assembly 14. The outer composite layer may also be PVA coated to ensure water/blood wettability to provide light transmittance during a procedure. Thus, in these embodiments, the outer composite balloon layer may define both a delivery subzone 16a and an activation subzone 16b coextensively.

When the inner composite balloon layer and the outer composite balloon layer are engaged together, they may form the cover layer 30 of the balloon assembly 14 of the integrated device 10, the inner and outer composite balloon layers defining coextensive delivery and activation subzones 16a, 16b, where the cover layer 30 being used in conjunction with an expansion layer 25 positioned axially interior to the cover layer 30. In those embodiments where a cover layer is not included, the inner composite balloon layer and the outer composite balloon layer are engaged together to form the balloon assembly 14. In both embodiments, the balloon assembly 14 is operable to both deliver treatment media (e.g., photoluminescent compounds including those previously disclosed, such as naphthalimide, riboflavin 5′-phosphate, or rose bengal) through the balloon assembly 14 and to transmit light in order to activate the treatment media. Such treatment media may be activated by light at various rates depending on the intensity and the length of exposure. Thus, as previously described, the integrated device 10 is operable to dilate a vessel, deliver treatment media, and to activate the treatment media without removing portions of the device from the delivery site or without breaking contact with the tissue wall.

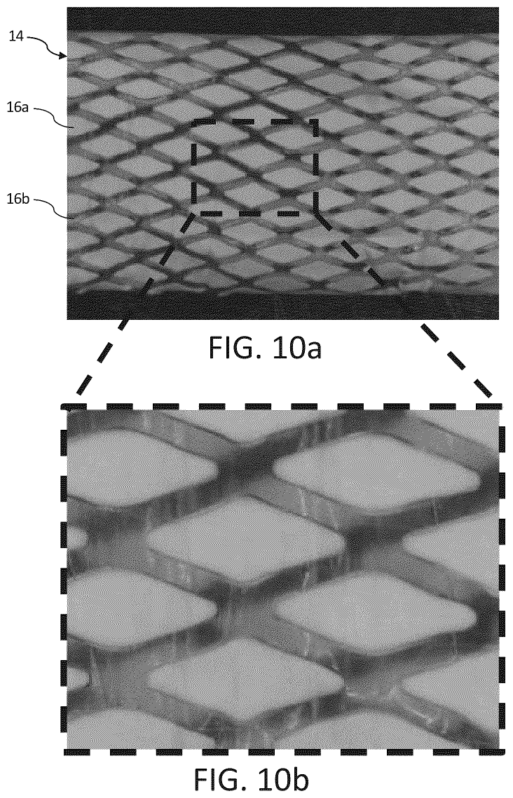

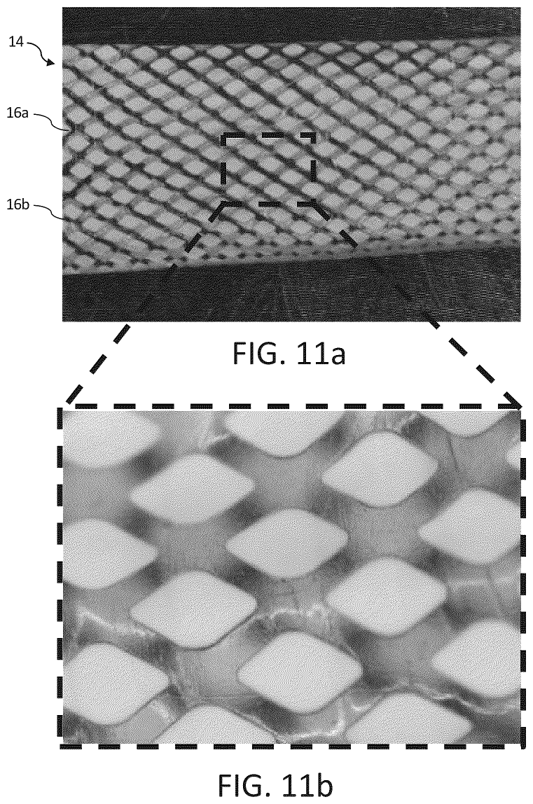

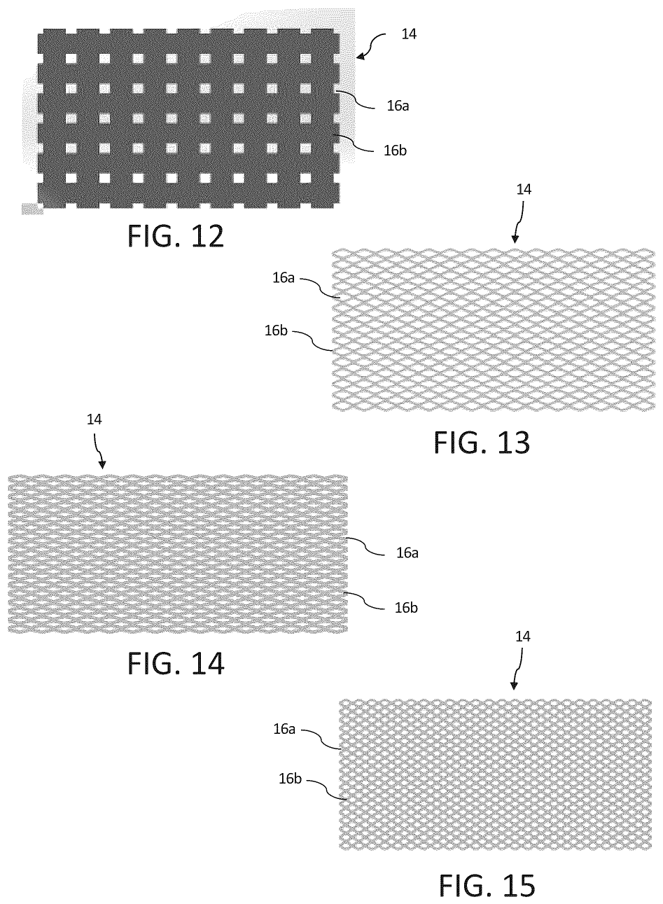

FIGS. 9-11b show various additional or alternative features of the treatment device 10. For example, a device 10 may include a material or layers of material (e.g., film) that is/are selected and/or modified to transfer fluids at a predetermined pressure or pressure ranges. Localized areas of the material may be densified to allow for light transmission, the densified areas defining the activation subzone 16b. Densification of the material may be accomplished via several processes, including, but not limited to, mechanical or thermal densification. During the densification process, for those areas of the material that have been densified, the material may have reduced or lost porosity at the densified regions, which reduces or prevents the densified zone from having a coextensive delivery subzone 16a and activation subzone 16b. The activation subzone 16b in this example includes the densified regions. In some embodiments, the densified regions may permit at least 40% light transmittance. In some embodiments, densified regions may permit at least 60% light transmittance. In some embodiments, the densified regions may permit at least 80% light transmittance.

The non-densified regions of the material allow for the weeping of the treatment medium while the densified regions allow for light transmittance. Thus, in this example the delivery subzone 16a includes the non-densified regions where weeping occurs. Various patterns of densification may be implemented in order optimize delivery and activation of treatment media. For example, a treatment media that requires a greater volume to be delivered for effective dosage may include a higher percentage of non-densified regions relative to densified regions. Conversely, a treatment media that requires longer exposure to, greater intensity of, or more direct exposure to light may be used with a balloon assembly 14 having a lower percentage of non-densified regions relative to densified regions. The relative percentage of densified and non-densified regions may be tailored to the specific requirements of the treatment media, the tissue to be treated, and other relevant factors.

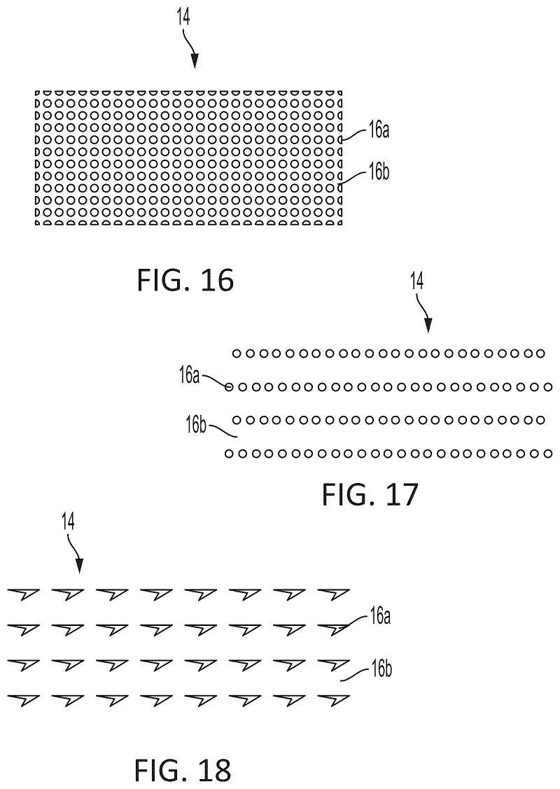

Various patterns of densification on the material may be implemented to provide corresponding zones for light transmission. For example, one pattern of densification may include a densification lattice defining non-densified regions, each substantially in the shape of a diamond. One diamond pattern can be seen in FIGS. 9-11 and 13. Another pattern of densification can produce non-densified regions, each substantially in the shape of squares. One square pattern can be seen in FIG. 12. Another pattern of densification can produce non-densified regions, each substantially in the shape of slits. One slit pattern can be seen in FIG. 14. Another pattern of densification can produce non-densified regions, each substantially in the shape of circles. One circle pattern can be seen in FIGS. 16-17. Another pattern of densification can produce non-densified regions, each substantially in any polygonal shape. One polygonal pattern can be seen in FIG. 18. It is within the scope of this disclosure that any number of patterns of densification can be implemented to for areas of densification and areas of non-densification in a variety of shapes and sizes, including polygonal and non-polygonal regions. It is also contemplated that the various regions may be non-uniform in shape or size across the material. For any of the examples provided, the densified regions and non-densified regions may be reversed in accordance with other embodiments. Furthermore, in any example, the patterns may be staggered, alternated, or modified.

The patterns and relative percentages of densified and non-densified regions may be adjusted in order to meet the requirements for uniform treatment media delivery to the target tissue and adequate light transmittance to the delivered treatment media. For example, the densified regions may comprise from about 40% to about 98% of the treatment zone 16 of the balloon assembly 14. In some embodiments the densified regions may comprise at least 40% of the treatment zone 16 of the balloon assembly 14. In some embodiments the densified regions may comprise at least 50% of the treatment zone 16 of the balloon assembly 14. In some embodiments the densified regions may comprise at least 60% of the treatment zone 16 of the balloon assembly 14. In some embodiments the densified regions may comprise at least 70% of the treatment zone 16 of the balloon assembly 14. In some embodiments the densified regions may comprise at least 80% of the treatment zone 16 of the balloon assembly 14. In some embodiments the densified regions may comprise at least 90% of the treatment zone 16 of the balloon assembly 14.

A range of values of light transmissivity may be obtained by preparing the materials used to form the treatment zone 16 in various ways. Furthermore, a combination of factors may be implemented to arrive at a desired value of light transmissivity while retaining the appropriate physical and material characteristics to retain structural integrity, conformity, and so forth. For example, the treatment zone 16 may be formed with delivery subzones 16a and activation subzones 16b, wherein the activation subzones 16b are tuned to a predefined light transmissivity by selecting the wrap angle of the materials, the number of layers of material used, the level of overwrap tension used, the number of cook cycles implemented, by using an underlayment layer in conjunction with a mandrel, the mandrel design including the use of patterned holes, and so forth. By varying these factors, the light transmissivity may be varied to obtain the specific light transmissivity desired.

In some embodiments, the treatment zone 16 may be densified such that the treatment zone 16 is operable to permit both delivery and activation through the balloon assembly 14 throughout the entire treatment zone 16. Stated otherwise, the treatment zone does not include separate subzones (e.g., patterns) of densified and non-densified regions. In an example, the treatment zone 16 may be formed of an ePTFE material that has been densified such that pores formed due to the node and fibril structure of the ePTFE at least partially remain in the treatment zone 16 and the treatment zones 16 are sufficiently light transmissive to allow at least a threshold amount of light to pass through at predetermined wavelengths in order to activate the treatment media (for example, when the treatment media is light activatable). Thus, the densification described that permits weeping and light transmission ubiquitously through the treatment zone 16 may be considered “intermediate densification”.

In some embodiments, wrapped, extruded and/or molded materials (e.g., film) form the cover layer 30 of the balloon assembly 14 of the integrated device 10, which is used in conjunction with an expansion layer 25 positioned axially interior to the cover layer 30. In those embodiments where a cover layer is not included, wrapped, extruded and/or molded materials form the balloon assembly 14, which is operable to dilate a vessel, deliver treatment media through the balloon assembly 14, and transmit light in order to activate the treatment media. Thus, as previously described, the integrated device 10 is operable to dilate a vessel, deliver treatment media, and to activate the treatment media without removing portions of the device from the delivery site or without breaking contact with the tissue wall.

One method for preparing a balloon assembly 14 having densified and non-densified regions includes wrapping extruded and/or molded materials over a patterned device. The patterned device may include, but is not limited to, a stent having patterns formed or laser-cut into or through the body of the stent. The patterned device may be used as a surface onto which the material is wrapped. The material may be helically wrapped to form the balloon assembly. When the material is helically wrapped, the helical wrapping may be at an angle from about 3 degrees to about 20 degrees. Thus, in various embodiments, the helical wrapping is at an angle of about 4 degrees, 5 degrees, 6 degrees, 7 degrees, 8 degrees, 8.3 degrees, 9 degrees, 10 degrees, 12 degrees, 15 degrees, or 20 degrees. Various material may be wrapped about the patterned device, including, but not limited to, ePTFE films of various porous and non-porous microstructures, composites including the ePTFE films with other various polymers and fluoropolymers in both continuous and discrete (e.g., patterned) combinations, ePTFE films and composited thereof with polyamide films, low WEP (e.g., water entry pressure from about 1-5 atm or approximately 2 atm), medium WEP (e.g., water entry pressure from about 5-14 or approximately 6 atm), high WEP (e.g., water entry pressure of about 14 atm or more), and open WEP materials (e.g., water entry pressure of about 0-1 atm), ePTFE films, and FEP films.

It is within the scope of this disclosure that any number of combinations of materials may be used in wrapping and any number of layers of wrapping may be implemented. Some layers may be wrapped at high forces. It is also within the scope of this disclosure that wrapping may also occur concentrically and that some layers may be wrapped helically and some concentrically. In some embodiments, portions of the material, either before being wrapped or after being wrapped may be modified, one example including portions of the material may be imbibed in order to provide areas of impermeability along the imbibed areas. For example, portions of the material may be imbibed with a tecothane solution. It will be noted that the disclosed method of wrapping and imbibing are not specific to this example but may be applied in other examples.

Once the material and/or materials and the desired layers or numbers of wraps have been applied to the patterned device, the patterned device and material may be heated or baked for a predetermined time at a predetermined temperature. Once the material has undergone curing, the material may be removed from the patterned device. In some embodiments the cured or baked material is everted when removed or after removal from the patterned device. The portions of the material that were in contact with the patterned device are the densified regions and operable to transmit therethrough. The non-densified portions are operable to allow the treatment media to pass therethrough.

In another embodiment, a device 10 may include a material that is tuned to weep at a predetermined pressure, wherein localized areas of the material may be doped or imbibed with a thermoplastic material (e.g., FEP, urethane, etc.) to allow for light transmission. The doped or imbibed areas are formed when the thermoplastic material contacts and is wicked through the material, which provides optical transparency (i.e., light transmissivity). The doped or imbibed areas define the activation subzone 16b One result of doping or imbibing the localized areas may include reduction or loss of porosity at the localized area. During the doping or imbibing process, for those areas of the material that have been imbibed, the material may have reduced or lost porosity at the imbibed regions, which reduces or prevents the imbibed zone from having a coextensive delivery subzone 16a and activation subzone 16b. The activation subzone 16b in this example includes the imbibed regions. In some embodiments, the imbibed regions may permit at least 40% light transmittance. In some embodiments, imbibed regions may permit at least 60% light transmittance. In some embodiments, the imbibed regions may permit at least 80% light transmittance.

The non-imbibed regions of the material allow for the weeping of the treatment medium while the imbibed regions allow for light transmittance. Thus, in this example the delivery subzone 16a includes the non-imbibed regions where weeping occurs. Various patterns of treatment may be implemented in order optimize delivery and activation of treatment media. For example, a treatment media that requires a greater volume to be delivered for effective dosage may include a higher percentage of non-imbibed regions relative to imbibed regions. Conversely, a treatment media that requires longer exposure to, greater intensity of, or more direct exposure to light may be used with a balloon assembly 14 having a lower percentage of non-imbibed regions relative to imbibed regions. The relative percentage of imbibed and non-imbibed regions may be tailored to the specific requirements of the treatment media, the tissue to be treated, and other relevant factors.

Various patterns of treatment of the wrapped, extruded and/or molded materials may be implemented to provide corresponding zones for light transmission. For example, one pattern of imbibed may include a lattice defining non-imbibed regions, each substantially in the shape of a diamond. Any of the patterns previously disclosed are also applicable to the present embodiments.

The patterns and relative percentages of imbibed and non-imbibed regions may be adjusted in order to meet the requirements for uniform treatment media delivery to the target tissue and adequate light transmittance to the delivered treatment media. In some embodiments, the wrapped, extruded and/or molded materials form the cover layer 30 of the balloon assembly 14 of the integrated device 10, which is used in conjunction with an expansion layer 25 positioned axially interior to the cover layer 30. In those embodiments where a cover layer is not included, the material forms the balloon assembly 14, which is operable to dilate a vessel, deliver treatment media through the balloon assembly 14, and transmit light in order to activate the treatment media. Thus, as previously described, the integrated device 10 is operable to dilate a vessel, deliver treatment media, and to activate the treatment media without removing portions of the device from the delivery site or without breaking contact with the tissue wall.

In some embodiments, additional material may be added to areas of the balloon assembly at which weeping should not occur during inflation (e.g., the shoulder 20).

In those embodiments in which a cover layer 30 and an expansion layer 25 are implemented, the expansion layer 25 may be formed using traditional balloon-forming methods. The cover layer 30 may include a non-compliant nylon or similar material (e.g., polyethylene, PET, PEBAX, or other semi-compliant materials) balloon that is operable to inflate to a predetermined diameter at a predetermined inflation pressure. The cover layer 30 may include a low WEP pressure PTFE film. The low WEP pressure PTFE film may be helically-wrapped or cigarette-wrapped, for example, or may be formed using other methods such as extrusion or molding. The cover layer 30 may be selected and/or modified to increase the threshold pressure at which the cover layer 30 weeps. Additionally, the cover layer 30 may be selected in order to assist in inflation resistance and/or diameter control of the balloon assembly 14. Specifically, when the cover layer 30 is selected to provide some inflation resistance and/or diameter control, the cover layer 30 is operable to provide increased consistency of weeping and controlled and consistent pressure of the intraluminal treatment media. The expansion layer 25 may be used to increase the pressure of the intraluminal treatment media, thereby pushing the treatment media through the delivery subzones 16a of the cover layer 30. One or both of the cover layer 30 and the expansion layer 25 may be light transmissive. In some embodiments, the cover layer 30 and/or the expansion layer 25 may permit at least 40% light transmittance. In some embodiments, the cover layer 30 and/or the expansion layer 25 may permit at least 60% light transmittance. In some embodiments, the cover layer 30 and/or the expansion layer 25 may permit at least 80% light transmittance. Thus, as previously described, the integrated device 10 is operable to dilate a vessel, deliver treatment media, and to activate the treatment media without removing portions of the device from the delivery site or without breaking contact with the tissue wall.

In those embodiments in which a cover layer 30 and an expansion layer 25 are implemented, the expansion layer 25 may be formed using traditional balloon-forming methods. The cover layer 30 may include a thin elastomeric urethane material that is operable to inflate to a predetermined diameter at a predetermined inflation pressure. The cover layer 30 may include expanded materials having retracted microstructures (e.g., bent or s-shaped fibrillated structures), including expanded retracted fluoropolymers such as ePTFE with or without secondary components (e.g., imbibed and/or coated fluoroelastomers) helically wrapped or may take other forms, such as concentrically wrapped or extruded forms. The cover layer 30 may provide mechanical structure that defines the diameter and length. Additionally, the cover layer 30 may weeps at a predetermined pressure. The expansion layer 25 is capable of inflation to angioplasty-level pressures and deflation. The cover layer 30 may be selected and/or modified to increase the threshold pressure at which the cover layer 30 weeps. The expansion layer 25 may be used to increase the pressure of the intraluminal treatment media, thereby pushing the treatment media through the delivery subzones 16a of the cover layer 30. One or both of the cover layer 30 and the expansion layer 25 may be light transmissive. In some embodiments, the cover layer 30 and/or the expansion layer 25 may permit at least 40% light transmittance. In some embodiments, the cover layer 30 and/or the expansion layer 25 may permit at least 60% light transmittance. In some embodiments, the cover layer 30 and/or the expansion layer 25 may permit at least 80% light transmittance. Thus, as previously described, the integrated device 10 is operable to dilate a vessel, deliver treatment media, and to activate the treatment media without removing portions of the device from the delivery site or without breaking contact with the tissue wall.

In another example, a balloon assembly 14 may be provided which is formed of expanded materials that have been densified, including expanded fluoropolymers such as ePTFE with or without secondary components (e.g., imbibed and/or coated fluoroelastomers) being at least 20% light transmissive (either wetted out or not) to form a structural member capable of maintaining a fixed diameter up to a predetermined pressure. Once the predetermined pressure has been achieved or exceeded, some pore structures through the balloon assembly 14 will be opened and/or augmented to form delivery subzones 16a and allow for weeping of the treatment media, while other portions are not opened and portions of the balloon assembly 14 remain unaugmented and retain light transmissive properties as activation subzones 16b that are, for example, light transmissive. Because the balloon assembly 14 is light transmissive, light can also be provided to activate the treatment media. In some embodiments, the balloon assembly 14 may permit at least 40% light transmittance. In some embodiments, the balloon assembly 14 may permit at least 60% light transmittance. In some embodiments, the balloon assembly 14 may permit at least 80% light transmittance. Thus, as previously described, the integrated device 10 is operable to dilate a vessel, deliver treatment media, and to activate the treatment media without removing portions of the device from the delivery site or without breaking contact with the tissue wall.

In accordance with the examples discussed above, a balloon assembly may include light blocking zones as previously disclosed. In some embodiments, the light blocking zones may be masked or otherwise modified or provided to allow from about 0% to about 5% light transmission.

A method of delivering a therapeutic compound is contemplated herein. The method includes providing an integrated device 10 a shaft 12 and a balloon assembly 14. The balloon assembly 14 includes a treatment zone 16 through which a therapeutic compound is delivered. The method includes filling the balloon assembly 14 with a fluid, for example, the therapeutic compound. The balloon assembly 14 is filled to a predefined pressure at which initial weep onset occurs. After initial weep onset occurs, the balloon assembly 14 is allowed to reach a settle pressure after initial weep, where the settle pressure is less than the pressure at which initial weep onset occurs. The pressure of the fluid in the balloon assembly 14 is then increased until the full weep threshold is obtained. The pressure may be maintained so as to permit sustained weeping. After sustained weeping, the introduction of fluid into the balloon assembly 14 may be decreased or stopped at which point the balloon assembly 14 may settle to a pressure. The balloon assembly 14 may then be pressurized back to a pressure prior to a highest sustainable pressure before weeping after sustained weeping. The highest sustainable pressure before weep allows for the balloon assembly 14 to be inflated and to be engaging the tissue, in some embodiments in a predefined shape, without further delivering fluid. At any point during the method, the fluid may be removed from the balloon assembly 14 and filled with a second fluid. For example, a first fluid may be a therapeutic compound and a second fluid may be a saline solution. The second fluid may be conducive to activation of the therapeutic compound that has been delivered to the target cited (e.g., via light activation).

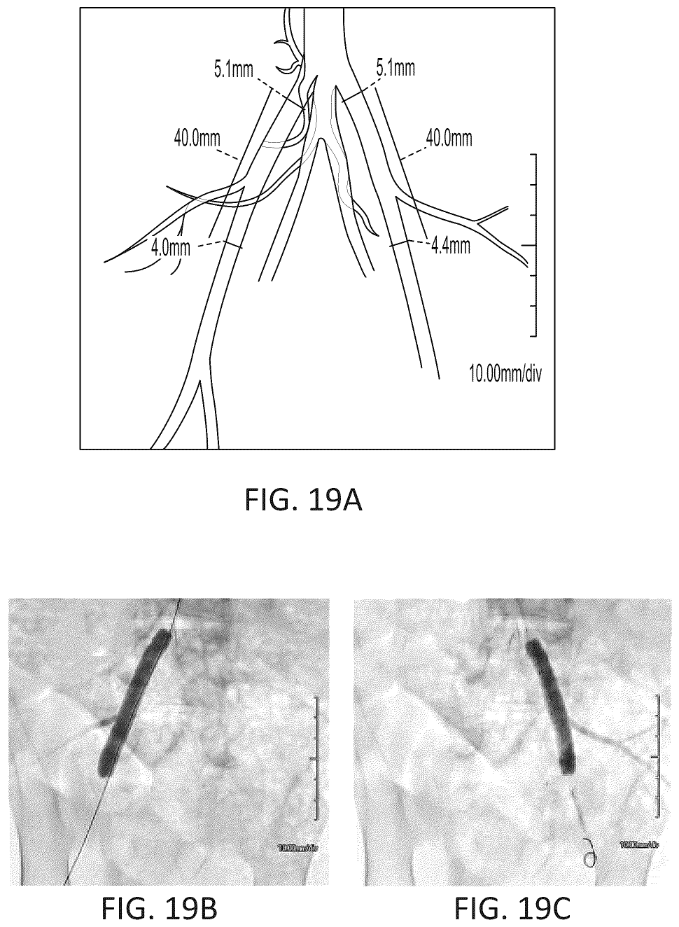

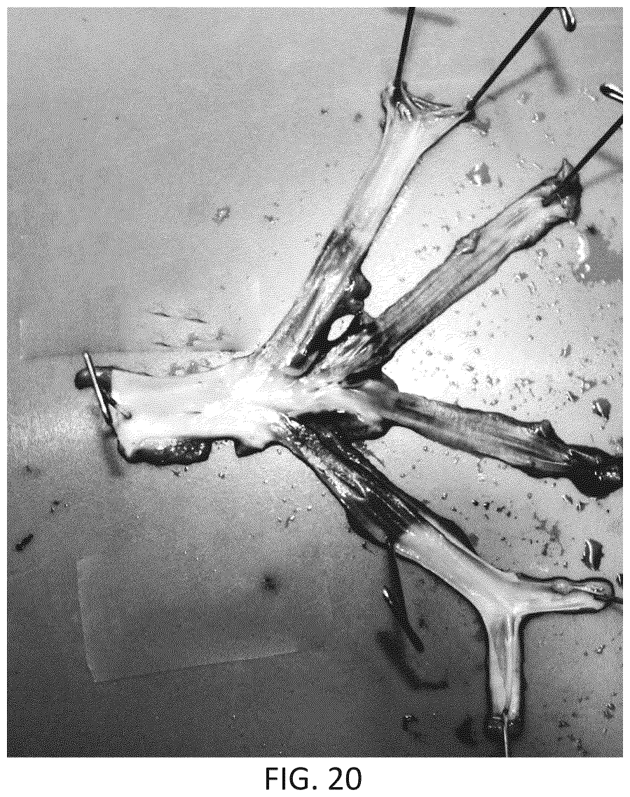

Referring to FIGS. 19A-19C and 20, an example of a method of delivering a fluid to a target tissue site is shown. FIG. 19A shows a pre-treatment angiogram of an iliac artery. The angiogram is used for sizing the balloon assembly 14 for use in treatment. FIGS. 19B and 19C show angiograms of branches of the artery as the balloon assembly 14 is positioned in that artery. The balloon assembly is inflated and, in some embodiments, weeping as shown. Contrast can be seen flowing down a collateral artery in FIGS. 19B and 19C, confirming the weeping of the contrast solution. As seen in FIG. 20, the iliac artery of FIGS. 19A-19C are shown in a necropsy. The blue dye-stained arteries are shown, where the blue dye was transmitted through the balloon assembly 14 and delivered to the tissue at the desired site.

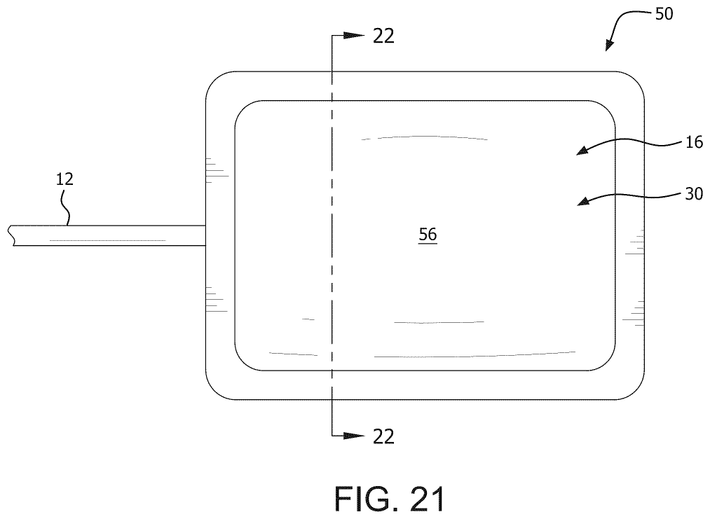

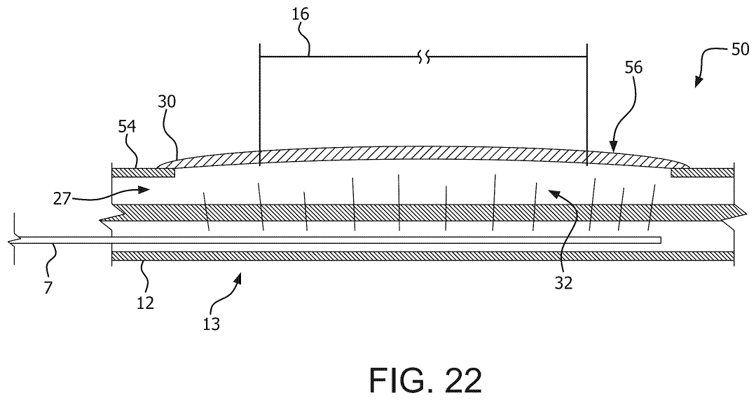

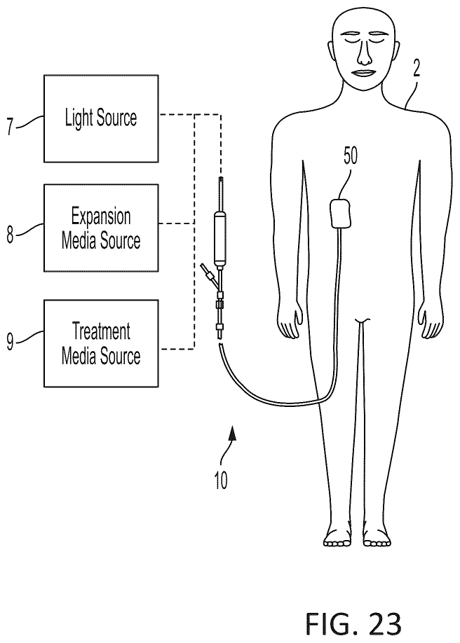

The previous embodiments are described in reference to a balloon assembly operable for delivering the treatment media to a vessel wall. It is appreciated that other form factors besides a balloon assembly are anticipated. In accordance with another embodiment, instead of a balloon assembly, the treatment device 10 includes a patch assembly 50. FIGS. 21 and 22 are a top view and cross-sectional view, respectively, of a patch assembly 50 in accordance with an embodiment. The patch assembly 50 defines a flat sheet that may be placed on broad surfaces of a patient 2, such as represented in FIG. 23. The treatment device 10, which is a fluid delivery system, is operable for delivering treatment media to a tissue of the patient. The treatment device 10 includes the patch assembly 50 which includes a delivery chamber 32 defining a wall 54, the wall 54 including a cover layer 30 that defines a planar treatment zone 16 having a porosity operable to allow controlled passage of a treatment media, which is a fluid, from the delivery chamber 32 to an outer surface 56 of the delivery chamber 32. The delivery chamber 32 is configured to deliver activatable treatment media to the treatment zone 16 of the delivery chamber 32 to transfer to the tissue of the patient and to activate the activatable treatment media at the tissue. The treatment device 10 includes a shaft 12 that defines a fluid channel 27 for supplying the delivery chamber 32 with the treatment media, and in accordance with another embodiment, for the placement of a light source 7 to illuminate and activate the treatment media that has passed through the treatment zone 16. As shown in FIG. 23, the treatment zone 16 of the patch assembly 50 may be placed on a surface of skin, and in other embodiments, organs, such as, but not limited to a heart wall or against the stomach wall, so as to place the treatment zone 16 in engagement with the tissue to be treated. The patch assembly 50 may be stiff or flexible, suitable for a particular purpose. A stiff patch assembly 50 may be suitable for urging engagement with tissue that may conform to the shape of the patch assembly 50, by way of example, but not limited thereto. Wherein a flexible patch assembly 50 may be suitable wherein the patch assembly 50 is required to conform to the shape of the tissue that is to be treated, by way of example, but not limited thereto. The activation light source 7 may illuminate the treatment site by passing light through the cover layer 30, the patch assembly 50, or after removal of the patch assembly 50 from the treatment site. In another embodiment, the patch assembly 50 further includes an inflation chamber adjacent the delivery chamber, the inflation chamber defined within an expansion layer, wherein the inflation chamber is configured to receive a pressurized expansion media for inflating the inflation chamber, wherein inflation of the inflation chamber is operable to urgingly engage the treatment zone against the tissue.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A treatment device for delivering a treatment to a tissue of a patient, comprising:
a patch assembly defining a flat sheet including a fluid delivery system including a delivery chamber defining a wall, the wall including a cover layer defining a planar treatment zone having a porosity operable to allow controlled passage of a fluid from the delivery chamber to an outer surface of the planar treatment zone, wherein the delivery chamber is configured to receive activatable treatment media and deliver the activatable treatment media to the treatment zone to transfer to the tissue of the patient, the delivery chamber operable to provide a conduit for light transmission operable to activate the activatable treatment media at the tissue.

2. The treatment device of claim 1, further comprising a light source in optical communication with the treatment zone of the delivery chamber.

3. The treatment device of claim 1, wherein at least a portion of the treatment zone is configured to be light transmissive.

4. The treatment device of claim 3, wherein the portion of the treatment zone configured to be light transmissive has a light transmissivity of at least 40% transmittance over a light wavelength range from 250 nm to 700 nm.

5. The treatment device of claim 1, wherein the delivery chamber includes an expansion layer.

6. The treatment device of claim 5, further comprising an inflation chamber adjacent the delivery chamber, the inflation chamber defined within the expansion layer, wherein the inflation chamber is configured to receive a pressurized expansion media for inflating the inflation chamber, wherein inflation of the inflation chamber is operable to urgingly engage the treatment zone against the tissue.

7. The treatment device of claim 5, wherein the delivery chamber is located between the expansion layer and the cover layer, wherein the delivery chamber is configured to receive the activatable treatment media for delivery to the tissue of the patient.

8. The treatment device of claim 7, wherein the delivery chamber is fluidically segregated from the inflation chamber.

9. The treatment device of claim 5, wherein the expansion layer includes a non-compliant material, a semi-compliant material, a compliant material, or combinations thereof.

10. The treatment device of claim 5, wherein the chamber further includes a delivery subzone and wherein the cover layer includes a material having porosity configured to weep the activatable treatment media from the delivery subzone when the activatable treatment media surpasses a threshold pressure within the delivery chamber.

11. The treatment device of claim 6, wherein the shaft includes an inflation conduit in fluid communication with the inflation chamber and a delivery conduit in fluid communication with the delivery chamber.

12. The treatment device of claim 1, wherein the activatable treatment media includes an extracellular matrix cross-linking promoter that is light activated.

13. The treatment device of claim 1, wherein the delivery chamber includes at least one layer of expanded polytetrafluoroethylene, polyethylene, polypelyne, or electrospun PTFE.

14. A method of providing treatment to a tissue of a patient's body, the method comprising:
positioning the treatment zone of the treatment device of claim 1 against the tissue of the patient's body;
delivering the activatable treatment media to the tissue of the patient at the treatment zone; and
activating the activatable treatment media at the tissue.
